(19) United States
(12) Patent Application Publication
(10) Pub. No.: US 2025/0205188 A1
(43) Pub. Date: Jun. 26, 2025

(54) TREATMENT OR PREVENTION OF RESPIRATORY DISEASES CHARACTERISED BY, OR INVOLVING, LUNG FIBROSIS

(71) Applicant: IMPERIAL COLLEGE INNOVATIONS LIMITED, London (GB)

(72) Inventors: Adam BYRNE, London (GB); Toby MAHER, St Albans, Hertfordshire (GB); Clare LLOYD, London (GB)

(21) Appl. No.: 18/847,581

(22) PCT Filed: Mar. 16, 2023
(86) PCT No.: PCT/GB2023/050629

§ 371 Date: Sep. 16, 2024

(30) Foreign Application Priority Data
Mar. 17, 2022 (GB) 2203755.0

(51) Int. Cl. A61K 31/225; A61K 9/00; A61K 9/127; A61K 31/194; A61K 31/197; A61K 45/06; A61P 11/00

(52) U.S. Cl. CPC A61K 31/225; A61K 9/007; A61K 9/127; A61K 31/194; A61K 31/197; A61K 45/06; A61P 11/00

(57) ABSTRACT

The present invention relates to compounds for use in the treatment and/or prevention of respiratory disease characterised by, or involving, lung fibrosis, as well as pharmaceutical compositions comprising the compounds. More specifically, the invention relates to compounds of Formula (I) described herein as inhibitors of succinate dehydrogenase for use in the treatment and/or prevention of lung fibrosis associated with a respiratory disease.

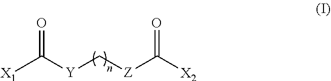

### FIELD OF THE INVENTION

The invention generally relates to compounds for use in the treatment and/or prevention of respiratory disease characterised by, or involving, lung fibrosis, as well as pharmaceutical compositions comprising the compounds. More specifically, the invention relates to compounds of Formula (I) described herein as inhibitors of succinate dehydrogenase for use in the treatment and/or prevention of lung fibrosis associated with a respiratory disease.

### BACKGROUND OF THE INVENTION

Idiopathic pulmonary fibrosis (IPF) is a chronic debilitating lung disease, characterized by the deposition of excessive extracellular matrix in the lung parenchyma. Existing pharmacological options are limited and with an increasing worldwide incidence and a median survival of 3 years from diagnosis, there is an urgent requirement to understand pathological mechanisms involved and to provide effective treatments.

A growing body of evidence supports a role for airway macrophages (AMs) in regulating pathogenic mechanisms underlying IPF. AMs are crucial in contributing to pulmonary defence, repair, surfactant processing and inflammatory responses. Moreover, AMs are strategically positioned at the interface between the airways and the environment and are found in the alveoli and airways, secreting numerous pro-fibrotic soluble mediators, chemokines, and matrix metalloproteases. Macrophages demonstrate remarkable plasticity and are capable of acquiring phenotypes which can both drive or resolve fibro-proliferative responses to injury. For example, AMs have been shown to be involved in the regulation of the extracellular matrix via secretion of matrix metalloproteases (MMPs) or by direct uptake of collagen.

Macrophage activation is tightly linked to cellular metabolism. Inflammatory activation of macrophages results in impaired mitochondrial respiration and tricarboxylic acid (TCA) cycle disruption, resulting in the accumulation of endogenous metabolites capable of adopting immunomodulatory roles. One such bioactive metabolite is itaconate. In macrophages, synthesis of itaconate is catalyzed by cis-aconitate decarboxylase (CAD), encoded by aconitate decarboxylase 1 (ACOD1), which mediates the decarboxylation of cis-aconitate to itaconate. Itaconate is one of the most highly induced metabolites in activated bone marrow-derived macrophages and can suppress the expression of pro-inflammatory cytokines. Furthermore, itaconate has been shown to control macrophage effector functions via competitive inhibition of succinate dehydrogenase (SDH) mediated oxidation of succinate and furthermore, drives an anti-inflammatory program via the KEAP-1-NRF2 axis. Therefore, itaconate appears to be a crucial regulator of macrophage phenotype and function. However, its functional significance in specialized tissue resident macrophages during chronic respiratory disease such as IPF remains unknown.

The inventors have previously linked AM phenotype to disease outcome in IPF, since increased numbers of AMs lacking the transferrin receptor CD71 are associated with worsened disease. The inventors have established that the ACOD1/itaconate axis is an endogenous pulmonary regulatory pathway, which limits fibrosis. Further, the inventors have observed that itaconate and cis-aconitate decarboxylase have therapeutic potential as targets in IPF and other diseases where fibrosis plays a role, particularly chronic respiratory diseases with a fibrotic component. In particular, the present inventors have demonstrated that the ACOD1/itaconate axis is altered in the human lung during IPF, that itaconate is an anti-fibrotic factor in the murine lung and that it impairs human fibroblast activity. The inventors have also shown that in patients with IPF, there is decreased expression of ACOD1 in AMs and reduced levels of airway itaconate, compared to healthy controls. ACOD1 deficiency in mice leads to more severe disease pathology in response to inhaled bleomycin, in comparison to wild-type (WT) controls, which is further exacerbated by adoptive transfer of ACOD1−/−, but not WT monocyte-recruited AMs. Addition of exogenous itaconate to cultures of human lung fibroblasts limits proliferation and wound healing and furthermore, inhaled itaconate (particularly oropharyngeal inhalation) ameliorates lung fibrosis in mice (Ogger et al., Sci Immunol., 5(52): doi:10.1126/sciimmunol.abc1884).

Mills et al., 2018, Nature, 556:113-117 have demonstrated that itaconate is a regulator of oxidative stress, through interaction with NRF2, a key anti-inflammatory pathway described for itaconate. When looking to provide alternative compounds having a comparable anti-fibrotic effect to that observed for itaconate, there remains a prevailing prejudice within the art that associates the impact of itaconate, at least partly, with its interaction with anti-inflammatory pathways (e.g. via the KEAP-1-NRF2 axis).

However, the inventors have surprisingly found that structural modifications to itaconate which would be expected to disrupt the interaction with NRF2, have been found not to negatively impact anti-fibrotic effects. Meanwhile, it has been shown by Ogger et al that anti-inflammatory effects associated with itaconate can in fact have a detrimental impact in the bleomycin mouse model of IPF, providing validation to previous work showing that treatment of IPF patients with a particular (anti-inflammatory) steroid combination therapy was severely detrimental to patient outcomes (N. Engl. J. Med. 2012; 366:1968-77).

Thus, the inventors have now been able to show definitively that the anti-fibrotic effects of itaconate are distinct from its well-known anti-inflammatory effects, thereby allowing the design and synthesis of itaconate analogue compounds for which anti-fibrotic effects may be maintained or enhanced, whilst anti-inflammatory effects may be diminished, in order to positively impact therapeutic outcomes. The present invention thus relates to compounds which possess anti-fibrotic effects (by virtue of an inhibitive effect upon succinate dehydrogenase) useful in the treatment of respiratory diseases characterised, or involving, lung fibrosis, including IPF.

### SUMMARY OF THE INVENTION

Accordingly, in a first aspect the invention provides a compound of Formula (I), or a pharmaceutically acceptable salt thereof, for use in treating or preventing a respiratory disease characterised by, or involving, lung fibrosis in a subject (“Compound For Use 1”),

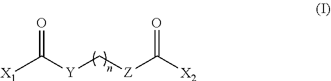

wherein:
Y is a direct bond, —CH═CH—, —NH—, or —O—;
   Z is —CH₂—, —C(═CR¹R²)—, —CR═CR—, —CH(R³)—, —CH(OH)—, —NR⁴—, —C(═O)—NR, —SO₂—NR—, —NH— or —O—;
X¹ is —OH, —OR⁴, or —NR⁵R⁶;
X² is —OH, —OR⁴, or —NR⁵R⁶;
R is independently H or a C₁₋₆-alkyl optionally substituted by one or more halogens;
R¹ and R² are independently H, a C₁₋₆-alkyl, C₁₋₆-alkyloxy, or C₁₋₃-alkyl.C₁₋₃-alkyloxy wherein alkyl, alkyloxy and alkyl.alkyloxy groups are optionally substituted by one or more halogens;
R³ is C₁₋₆-alkyl, C₁₋₆-alkyloxy, or C₁₋₃-alkyl.C₁₋₃-alkyloxy, optionally substituted by one or more halogens;
R⁴ is independently C₁₋₁₂-alkyl, or C₁₋₆-alkyl.C₁₋₆-alkyloxy, optionally substituted by one or more halogens;
R⁵ and R⁶ are independently H, C₁₋₁₂-alkyl, or C₁₋₆alkyl.C₁₋₆alkyloxy, wherein alkyl and alkyl-alkyloxy groups are optionally substituted by one or more halogens;
   n is 0 to 4
with the following provisos:
   1) when Y is —CH═CH—, Z is not —CH₂—;
   2) when Y is —CH═CH— and Z is —CR═CR—, n is not 0;
   3) when Y is a direct bond, Z is —C(═CH₂)— and n is 1, at least one of X¹ and X² is other than —OH;
   4) when Y is a direct bond, Z is —CH₂— and n is 0 or 1, at least one of X¹ and X² is —NR⁵R⁶; and
   5) when Y is a direct bond, Z is —CH═CH— and n is 0, at least one of X¹ and X² is —NR⁵R⁶.

In some embodiments, at least one of X¹ and X² is —OR⁴, or —NR⁵R⁶, preferably at least one of X¹ and X² is —OR⁴.

In some embodiments, wherein n is 0 to 2, preferably wherein n is 0 or 1.

In some embodiments, Y is a direct bond or —CH═CH—, preferably wherein Y is a direct bond.

In some embodiments, Z is —CH₂—, —C(═CR¹R²)—, —CH(R³)—, —CH(OH)—, or —NR⁴—.

In some embodiments, Z is —C(═CR¹R²)— or —CH(R³)—.

In some embodiments, Z is —C(═CR¹R²)— and wherein at least one, preferably both, of R¹ and R² is/are H.

In some embodiments, Z is —C(═CR¹R²)— and one of R¹ and R² is C₁-C₄ alkyl, preferably wherein one of R¹ and R² is methyl, optionally substituted by one or more halogens.

In some embodiments, Z is —CR═CR— and at least one R group, preferably both, is/are H.

In some embodiments, Z is —CR═CR— and one R group is C₁-C₄ alkyl, preferably methyl, optionally substituted with one or more halogens and one R group is H.

In some embodiments, at least one of R⁵ and R⁶, preferably both, are C₁-C₁₂ alkyl, optionally substituted with one or more halogens.

In some embodiments, R⁴ is C₁-C₁₂ alkyl, optionally substituted with one or more halogens.

In some embodiments, at least one of R⁵ and R⁶, preferably both, are C₁-C₆ alkyl (e.g. methyl, iso-propyl or t-butyl), optionally substituted with one or more halogens; In some embodiments, R⁴ is C₁-C₆ alkyl (e.g. methyl, iso-propyl or t-butyl), optionally substituted with one or more halogens.

In some embodiments, Y is a direct bond, Z is —CR═CR—, n is 0, and at least one R group, preferably both groups, is/are H, and wherein when one R group is other than H, the R group is C₁-C₆ alkyl (e.g. methyl, iso-propyl or t-butyl), and preferably wherein the X¹—C(═O)— moiety is trans to the —C(═O)—X² moiety.

In some embodiments, where Y is a direct bond, Z is —CH(OH)—, and n is 1.

In some embodiments, the compound is selected from:

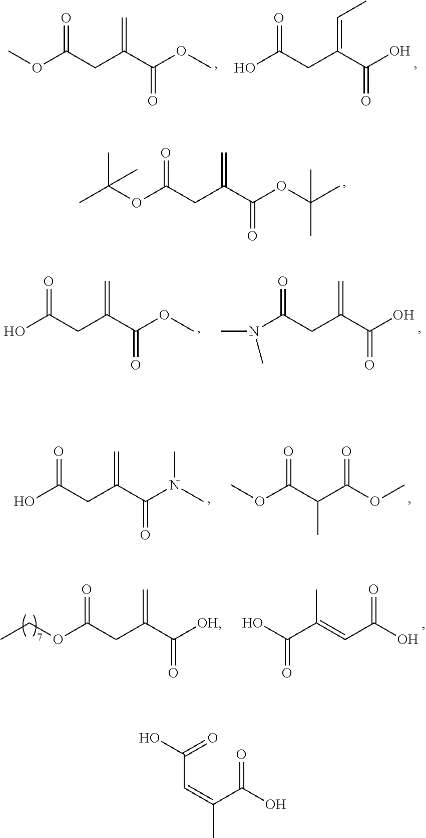

or a pharmaceutically acceptable salt thereof.

The compound of Formula (I), or a pharmaceutically acceptable salt thereof, may directly inhibit succinate dehydrogenase.

The compound of Formula (I), or a pharmaceutically acceptable salt thereof, for use in the treatment or prevention of a respiratory disease characterised by, or involving, lung fibrosis may be administered by: inhalation; intraperitoneal, subcutaneous, and/or intramuscular injection; infusion; and/or orally, preferably wherein the compound of Formula (I), or a pharmaceutically acceptable salt thereof, is administered by oropharyngeal inhalation and/or nasal inhalation.

The compound of Formula (I), or a pharmaceutically acceptable salt thereof, for use in the treatment or prevention of a respiratory disease characterised by, or involving, lung fibrosis may be delivered in a drug delivery system, wherein optionally said drug delivery system is a liposome-based drug delivery system.

The compound of Formula (I), or pharmaceutically acceptable salt thereof, may be administered at a dose of about 0.1 mg/kg to about 10 mg/kg.

The compound of Formula (I), or a pharmaceutically acceptable salt thereof, may be administered once per week to about four times per day, preferably about once per day.

According to the present invention, the treatment or prevention may modify the metabolic and/or fibrotic phenotype of tissue-resident macrophages (Tr-Ms), preferably wherein the treatment or prevention increases the metabolic phenotype and/or reduces the fibrotic phenotype of the Tr-M.

The treatment or prevention may increase the proportion of CD11b⁺/MHCII⁺ Tr-Ms resident in the lung tissue.

The treatment or prevention may modify the metabolic and/or fibrotic phenotype of fibroblasts within the lung tissue, preferably wherein the treatment or prevention reduces the metabolic and/or fibrotic phenotype of the fibroblasts.

The treatment or prevention may: reduce the oxygen consumption rate, maximal respiration and/or spare respiratory capacity of fibroblasts; reduce proliferation of fibroblasts; and/or reduce the wound healing capacity of fibroblasts.

The treatment or prevention may result in: an improvement in the fibrosis of the lung tissue; a decrease in lung tissue collagen expression, preferably Col3α1, Col1α1 and/or Col4α1; a decrease in lung tissue fibronectin (Fn1) expression; a decrease in IL-1β expression in fibroblasts obtained from the lung tissue; and/or a decrease in hydroxyproline levels.

According to the invention, the fibrosis is in lung tissue, wherein the fibrosis may be a characteristic of a particular respiratory disease, or otherwise involved in a respiratory disease. The respiratory disease may be pulmonary fibrosis, wherein the pulmonary fibrosis is any form of chronic fibrosing interstitial lung disease including idiopathic pulmonary fibrosis. The respiratory disease may also include forms of asthma which have given rise to airway fibrosis (e.g. airway subepithelial fibrosis).

The treatment or prevention according to the invention may result in: a) an improvement in lung function, preferably an increase in forced vital capacity, an increase in total lung capacity and/or an increase in the transfer capacity of the lung for the uptake of carbon monoxide, as measured by gas transfer (TLco) test; b) a reduction in the decline of forced vital capacity; c) preservation or improvement of exercise capacity; d) a reduction in the progression of fibrosis as quantified by high resolution computed tomography; e) preservation or improvement of quality of life; and/or (f) improved survival.

In another aspect, the present invention relates to methods of treatment or prevention of a respiratory disease characterised by, or involving, lung fibrosis in a subject, said method comprising administering a compound of Formula (I), or a pharmaceutically acceptable salt thereof, to the subject.

In another aspect, the present invention provides a pharmaceutical composition comprising a compound of Formula (I), or pharmaceutically acceptable salt thereof, as described herein, wherein the pharmaceutical composition is adapted for administration by oropharyngeal inhalation and/or nasal inhalation; and/or incorporated into a liposome-based drug delivery system.

### BRIEF DESCRIPTION OF THE DRAWINGS

FIG. 1: shows the relative rate of wound healing in human epithelial cells which have been subjected to 0.1 mM of compounds of Formula (I)-(A) relative wound closure overtime of epithelial cells (rate calculated using an ImageJ/Fiji® plugin by Suarez-Arnedo et al.); (B) relative wound closure of wound after 4 hours; and (C) relative wound closure of wound after 8 hours.

FIG. 2: show the results of gene expression analysis in healthy and fibrosis modelled human fibroblast cells treated with 10 mM of compounds of Formula (I) and comparative compounds, including itaconate—(A) gene expression of COL1A1 relative to two housekeeping genes (bactin and Beta-2-Microglobulin-averaged to make a ‘pooled’ control); (B) gene expression of FN1 relative to housekeeping gene; and (C) gene expression of CTGF relative to housekeeping gene.

FIG. 3: shows the results of the therapeutic administration of dimethyl malonate (DMM)-liposomes, illustrating the benefit of the liposomal delivery system in improving lung function in murine bleomycin model of lung fibrosis by demonstrating compliance at the base line of murine lung function when treated with dimethyl-malonate encapsulated liposomes.

FIG. 4: shows the results of a glycolytic stress test (Seahorse assay) with healthy primary human lung fibroblasts incubated with different concentrations of the itaconate analogue, citraconate, in terms of the effect on Extracellular Acidification Rate (ECAR) (indicative of glycolytic activity).

FIG. 5: shows the results of a glycolytic stress test (Seahorse assay) with healthy primary human lung fibroblasts incubated with different concentrations of the itaconate analogue, mesaconate, in terms of the effect on Extracellular Acidification Rate ECAR (indicative of glycolytic activity).

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The various embodiments described herein can be combined to provide further embodiments. These and other changes can be made to the disclosure in light of the detailed description. All such modifications are intended to be included within the scope of the appended claims.

The headings provided herein are not limitations of the various aspects or embodiments of this disclosure.

As used herein, the term “C₁₋₆alkyl” means a saturated linear or branched free radical consisting essentially of 1 to 6 carbon atoms and a corresponding number of hydrogen atoms. Exemplary C₁₋₆alkyl groups include methyl, ethyl, n-propyl, isopropyl, n-butyl, isobutyl, etc. Other C₁₋₆alkyl groups will be readily apparent to those of skill in the art given the benefit of the present disclosure. The terms “C₁₋₄-alkyl”, “C₁₋₁₂-alkyl”, etc., have equivalent meanings, i.e. saturated linear or branched free radical consisting essentially of 1 to 4 (or 12) carbon atoms and a corresponding number of hydrogen atoms.

As used herein, the term “C₁₋₆alkyloxy” means a saturated linear or branched free radical consisting essentially of 1 to 6 carbon atoms (and a corresponding number of hydrogen atoms) and an oxygen atom. A C₁₋₆alkyloxy group is attached via the oxygen atom. Exemplary C₁₋₆alkyloxy groups include methyloxy, ethyloxy, n-propyloxy, isopropyloxy, n-butyloxy, isobutyloxy, etc. Other C₁₋₆alkyloxy groups will be readily apparent to those of skill in the art given the benefit of the present disclosure. The terms “C₁₋₃-alkyloxy”, “C₁₋₄-alkyloxy”, and the like, have an equivalent meaning, i.e. a saturated linear or branched free radical consisting essentially of 1 to 3) carbon atoms and a corresponding number of hydrogen atoms saturated linear or branched free radical consisting essentially of 1 to 3 (or 4) carbon atoms (and a corresponding number of hydrogen atoms) and an oxygen atom, wherein the group is attached via the oxygen atom.

As used herein, the “halogen” means a fluorine, chlorine, bromine, or iodine free radical group.

As used herein, the term “C₁₋₆alkyl-C₁₋₆alkyloxy” means a saturated linear or branched free radical consisting essentially of 1 to 6 carbon atoms and a corresponding number of hydrogen atoms (“C₁₋₆alkyl”) bonded to a saturated linear or branched free radical consisting essentially of 1 to 6 carbon atoms (and a corresponding number of hydrogen atoms) and an oxygen atom (“C₁₋₆alkyloxy”), wherein the alkyl and alkyloxy groups are bonded via the oxygen atom of the alkyloxy. The term “C₁₋₃-alkyl-C₁₋₃-alkyloxy”, and the like, has an equivalent meaning, i.e. a saturated linear or branched free radical consisting essentially of 1 to 3 carbon atoms and a corresponding number of hydrogen atoms (“C₁₋₃-alkyl”) bonded to a saturated linear or branched free radical consisting essentially of 1 to 3 carbon atoms (and a corresponding number of hydrogen atoms) and an oxygen atom (“C₁₋₃-alkyloxy”), wherein the alkyl and alkyloxy groups are bonded via the oxygen atom of the alkyloxy.

The recitation of a listing of chemical groups in any definition of a variable herein includes definitions of that variable as any single group or combination of listed groups.

The recitation of an embodiment for a variable or aspect herein includes that embodiment as any single embodiment or in combination with any other embodiments or portions thereof.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

References herein to the level of a particular molecule (e.g. a compound of Formula (I), or a pharmaceutically acceptable salt thereof) encompass the actual amount of the molecule, such as the mass, molar amount, concentration or molarity of the molecule. Preferably in the context of the invention, references to the level of a particular molecule refer to the concentration of the molecule.

The level of a molecule may be determined in any appropriate physiological compartment. Preferred physiological compartments include bronchoalveolar lavage (BAL), plasma, whole blood and/or serum. The level of a molecule may be determined from any appropriate sample from an individual, e.g. a BAL sample plasma sample, a blood sample and/or a serum sample. Other non-limiting examples of samples which may be tested are tissue or fluid samples urine and biopsy samples. Thus, by way of non-limiting example, the invention may reference the level (e.g. concentration) of a molecule (e.g. a compound of Formula (I)) in the BAL and/or plasma of an individual.

The level of a molecule (e.g. itaconate, collagen, fibrinogen and/or hydroxyproline) may be compared with any appropriate control. For example, a control may be obtained from a healthy individual or an individual without (clinically relevant) fibrosis in the tissue to be treated according to the invention. Alternatively, the control may be obtained from the same individual prior to treatment, or from a different individual with (clinically relevant) fibrosis in the same tissue type as to be treated, but wherein the different individual has not been treated with a compound of Formula (I), or a pharmaceutically acceptable salt thereof, according to the invention.

The level of a molecule (e.g. itaconate, collagen, fibrinogen and/or hydroxyproline) after treatment with a compound of Formula (I), or a pharmaceutically acceptable salt thereof, may be compared with the level of the molecule in the individual pre-treatment with the agent. Thus, the invention may be concerned with the relative level of the molecule (e.g. itaconate, collagen, fibrinogen and/or hydroxyproline) pre- and post-treatment. The level of a molecule pre-treatment (e.g. itaconate, collagen, fibrinogen and/or hydroxyproline) may be used to identify an individual as suitable for treatment according to the invention. Other parameters may also be used, either alone or in combination with the level of a molecule as described above, to identify an individual as suitable for treatment according to the invention. Suitable parameters to identify an individual as suitable for treatment according to the invention are known to the skilled person. Typically, the presence and/or amount of a biomarker is used to identify an individual as suitable for treatment. The biomarker may, for example, be a circulating protein biomarker. The circulating biomarker may be a epithelial cell damage marker (e.g. cytokeratin 19 fragment (CYFRA 21-1) or carbohydrate antigen 125 (CA125)), a marker of collagen turnover (e.g. procollagen type I N-terminal propeptide (PINP); procollagen type I C-terminal propeptide (PICP); carboxyl-terminal peptide of procollagen type I (PIP); and carboxyl-terminal telopeptide of collagen type I (CITP)), or a marker of macrophage function (e.g. YKL40, CCL18 and PA11). Imaging biomarkers may also be used to identify an individual as suitable for treatment according to the invention. The imaging biomarkers may be identified, for example, through analysis of computerised tomography (CT) images.

The term “itaconate” as used herein refers to 2-methylidenebutanedioic acid. Various synonyms of itaconate are known to the skilled person including, itaconic acid, 2-methylenesuccinic acid, 2-propene-1,2-dicarboxylic acid, methylenebutanedioic acid, methylenesuccinic acid, and propylenedicarboxylic acid, which are all encompassed by the term “itaconate”. Itaconate has been assigned Chemical Abstracts Service registry number (CAS No.) 97-65-4.

The level of a molecule may be measured directly or indirectly, and may be determined using any appropriate technique. Suitable standard techniques are known in the art, for example Western blotting and enzyme-linked immunosorbent assays (ELISAs).

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like.

A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionisable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts”. The term “pharmaceutically-acceptable salt” refers to salts which possess toxicity profiles within a range that affords utility in pharmaceutical applications.

Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from, more preferably, an inorganic acid or, less preferably, from an organic acid. Examples of inorganic acids include hydrochloric, hydrobromic, hydriodic, nitric, carbonic, sulfuric, and phosphoric acids. Examples of organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafiuoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts.

Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The publications discussed herein are provided solely for their disclosure prior to the filing date of the present application. Nothing herein is to be construed as an admission that such publications constitute prior art to the claims appended hereto.

Compounds of Formula (I), and Pharmaceutically Acceptable Salts The present invention provides a compound of Formula (I), or a pharmaceutically acceptable salt thereof, for use in treating or preventing a respiratory disease characterised by, or involving, lung fibrosis in a subject (“Compound For Use 1”),

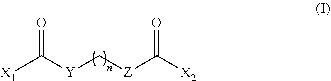

wherein:
Y is a direct bond, —CH═CH—, —NH—, or —O—;
   Z is —CH₂—, —C(═CR¹R²)—, —CR═CR—, —CH(R³)—, —CH(OH)—, —NR⁴—, —C(═O)—NR, —SO₂—NR—, —NH— or —O—;
X¹ is —OH, —OR⁴, or —NR⁵R⁶;
X² is —OH, —OR⁴, or —NR⁵R⁶;
R is independently H or a C₁₋₆-alkyl optionally substituted by one or more halogens;
R¹ and R² are independently H, a C₁₋₆-alkyl, C₁₋₆-alkyloxy, or C₁₋₃-alkyl.C₁₋₃-alkyloxy wherein alkyl, alkyloxy and alkyl.alkyloxy groups are optionally substituted by one or more halogens;
R³ is C₁₋₆-alkyl, C₁₋₆-alkyloxy, or C₁₋₃-alkyl.C₁₋₃-alkyloxy, optionally substituted by one or more halogens;
R⁴ is independently C₁₋₁₂-alkyl, or C₁₋₆-alkyl.C₁₋₆-alkyloxy, optionally substituted by one or more halogens;
R⁵ and R⁶ are independently H, C₁₋₁₂-alkyl, or C₁₋₆alkyl.C₁₋₆alkyloxy, wherein alkyl and alkyl-alkyloxy groups are optionally substituted by one or more halogens;
   n is 0 to 4
with the following provisos:
   6) when Y is —CH═CH—, Z is not —CH₂—;
   7) when Y is —CH═CH— and Z is —CR═CR—, n is not 0;
   8) when Y is a direct bond, Z is —C(═CH₂)— and n is 1, at least one of X¹ and X² is other than —OH;
   9) when Y is a direct bond, Z is —CH₂— and n is 0 or 1, at least one of X¹ and X² is —NR⁵R⁶; and
   10) when Y is a direct bond, Z is —CH═CH— and n is 0, at least one of X¹ and X² is —NR⁵R⁶.

In some embodiments, at least one of X¹ and X² is —OR⁴, or —NR⁵R⁶, preferably at least one of X¹ and X² is —OR⁴.

In some embodiments, wherein n is 0 to 2, preferably wherein n is 0 or 1.

In some embodiments, Y is a direct bond or —CH═CH—, preferably wherein Y is a direct bond.

In some embodiments, Z is —CH₂—, —C(═CR¹R²)—, —CH(R³)—, —CH(OH)—, or —NR⁴—.

In some embodiments, Z is —C(═CR¹R²)— or —CH(R³)—.

In some embodiments, Z is —C(═CR¹R²)— and wherein at least one, preferably both, of R¹ and R² is/are H.

In some embodiments, Z is —C(═CR¹R²)— and one of R¹ and R² is C₁-C₄ alkyl, preferably wherein one of R¹ and R² is methyl, optionally substituted by one or more halogens.

In some embodiments, Z is —CR═CR— and at least one R group, preferably both, is/are H.

In some embodiments, Z is —CR═CR— and one R group is C₁-C₄ alkyl, preferably methyl, optionally substituted with one or more halogens and one R group is H.

In some embodiments, at least one of R⁵ and R⁶, preferably both, are C₁-C₁₂ alkyl, optionally substituted with one or more halogens.

In some embodiments, R⁴ is C₁-C₁₂ alkyl, optionally substituted with one or more halogens.

In some embodiments, at least one of R⁵ and R⁶, preferably both, are C₁-C₆ alkyl (e.g. methyl, iso-propyl or t-butyl), optionally substituted with one or more halogens; In some embodiments, R⁴ is C₁-C₆ alkyl (e.g. methyl, iso-propyl or t-butyl), optionally substituted with one or more halogens.

In some embodiments, Y is a direct bond, Z is —CR═CR—, n is 0, and at least one R group, preferably both groups, is/are H, and wherein when one R group is other than H, the R group is C₁-C₆ alkyl (e.g. methyl, iso-propyl or t-butyl), and preferably wherein the X¹—C(═O)— moiety is trans to the —C(═O)—X² moiety.

In some embodiments, where Y is a direct bond, Z is —CH(OH)—, and n is 1.

In some embodiments, the compound is selected from:

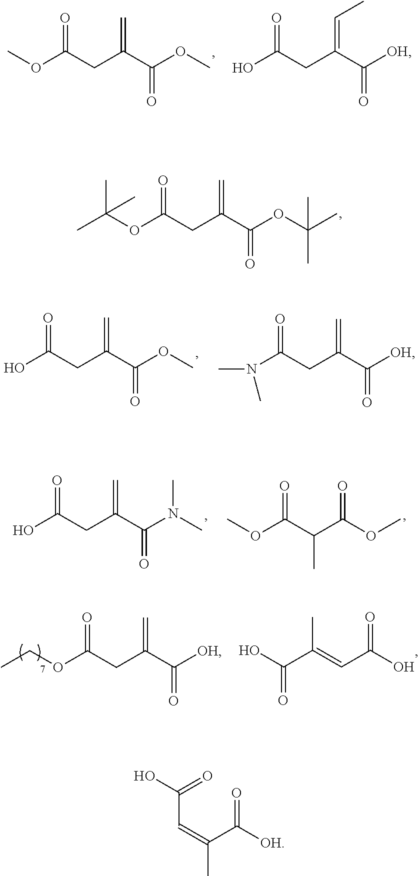

or a pharmaceutically acceptable salt thereof.

It will be understood that when the compounds of Formula (I), or pharmaceutically acceptable salts thereof, contain one or more chiral centres, the compounds may exist in, and may be isolated as pure enantiomeric or diastereomeric forms or as racemic mixtures. The present invention therefore includes any possible enantiomers, diastereomers, racemates or mixtures thereof of the compounds of Formula (I), or pharmaceutically acceptable salts thereof.

The compounds of Formula (I), or a pharmaceutically acceptable salts thereof, may have rotameric forms, or may not have rotational activity. Rotameric forms include slow rotating forms and fast rotating forms. In some preferred embodiments, fast rotating forms of the compounds of Formula (I), or a pharmaceutically acceptable salts thereof, are preferred.

The compounds of Formula (I), or a pharmaceutically acceptable salts thereof, may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. The invention encompasses any tautomeric form of a compound of Formula (I), or a pharmaceutically acceptable salt thereof, and is not to be limited merely to any one tautomeric form. Thus, compounds of Formula (I), or a pharmaceutically acceptable salts thereof, described herein encompass tautomers (including keto-enol and amide-imidic acid forms).

Pharmaceutically acceptable hydrates, solvates, polymorphs, etc., of the compounds of Formula (I) described herein are within the scope of the present disclosure. Compounds of Formula (I), or pharmaceutically acceptable salts thereof, as described herein may be in an amorphous form and/or in one or more crystalline forms.

Isotopically-labeled compounds are also within the scope of the present disclosure. As used herein, an “isotopically-labeled compound” refers to a presently disclosed compound including pharmaceutically acceptable salts thereof, as described herein, in which one or more atoms are replaced by an atom having an atomic mass or mass number different from the atomic mass or mass number usually found in nature.

Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

The present invention will now be described by means of the following numbered embodiments:

Embodiment 1.1: Compound For Use 1, wherein n is 0.

Embodiment 1.2: Compound For Use 1, wherein n is 1.

Embodiment 1.3: Compound For Use 1, wherein n is 2.

Embodiment 1.4: Compound For Use 1, wherein n is 3.

Embodiment 1.5: Compound For Use 1, wherein n is 4.

Embodiment 1.6: Compound For Use 1, wherein n is 0 to 2.

Embodiment 1.7: Compound For Use 1, wherein n is 0 or 1.

Embodiment 1.8: Compound For Use 1, or any of Embodiments 1.1 to 1.7, wherein X¹ is —OR⁴, or —NR⁵R⁶.

Embodiment 1.9: Compound For Use 1, or any of Embodiments 1.1 to 1.7, wherein X¹ is —OH, or —OR⁴.

Embodiment 1.10: Compound For Use 1, or any of Embodiments 1.1 to 1.7, wherein X¹ is —OH, or —NR⁵R⁶.

Embodiment 1.11: Compound For Use 1, or any of Embodiments 1.1 to 1.7, wherein X¹ is —OH.

Embodiment 1.12: Compound For Use 1, or any of Embodiments 1.1 to 1.7, wherein X¹ is —OR⁴.

Embodiment 1.13: Compound For Use 1, or any of Embodiments 1.1 to 1.7, wherein X¹ is —NR⁵R⁶.

Embodiment 1.14: Compound For Use 1, or any of Embodiments 1.1 to 1.7, wherein X¹ and X². are both —OH.

Embodiment 1.15: Compound For Use 1, or any of Embodiments 1.1 to 1.14, wherein X² is —OH, or —OR⁴.

Embodiment 1.16: Compound For Use 1, or any of Embodiments 1.1 to 1.14, wherein X² is —OH, or —NR⁵R⁶.

Embodiment 1.17: Compound For Use 1, or any of Embodiments 1.1 to 1.14, wherein X² is —OH.

Embodiment 1.18: Compound For Use 1, or any of Embodiments 1.1 to 1.14, wherein X² is —OR⁴.

Embodiment 1.19: Compound For Use 1, or any of Embodiments 1.1 to 1.14, wherein X² is —NR⁵R⁶.

Embodiment 1.20: Compound For Use 1, or any of Embodiments 1.1 to 1.19, wherein X¹ and X² are selected from the same group.

Embodiment 1.21: Compound For Use 1, or Embodiment 1.20, wherein X¹ and X². are both independently selected from —OH.

Embodiment 1.22: Compound For Use 1, or Embodiment 1.20, wherein X¹ and X². are both independently selected from —OR⁴.

Embodiment 1.23: Compound For Use 1, or Embodiment 1.20, wherein X¹ and X². are both independently selected from —NR⁵R⁶.

Embodiment 1.24: Compound For Use 1, or any of Embodiments 1.1 to 1.19, wherein X¹ and X² are selected from different groups.

Embodiment 1.25: Compound For Use 1, or Embodiment 1.24, wherein X¹ is —OH and X² is —OR⁴.

Embodiment 1.26: Compound For Use 1, or Embodiment 1.24, wherein X¹ is —OH and X² is NR⁵R⁶.

Embodiment 1.27: Compound For Use 1, or Embodiment 1.24, wherein X¹ is —OR⁴ and X² is —OH.

Embodiment 1.28: Compound For Use 1, or Embodiment 1.24, wherein X¹ is —OR⁴ and X² is —NR⁵R⁶.

Embodiment 1.29: Compound For Use 1, or Embodiment 1.24, wherein X¹ is —NR⁵R⁶ and X² is —OH.

Embodiment 1.30: Compound For Use 1, or Embodiment 1.24, wherein X¹ is —NR⁵R⁶ and X² is —OR⁴.

Embodiment 1.31: Compound For Use 1, or any one of Embodiments 1.12, 1.18, 1.25, 1.27, 1.28 and 1.30, wherein R⁴ is independently C₁₋₁₂-alkyl optionally substituted by one or more halogens.

Embodiment 1.32: Compound For Use 1, or Embodiments 1.12, 1.18 or 1.31, wherein R⁴ is independently C₁₋₈alkyl optionally substituted by one or more halogens.

Embodiment 1.33: Compound For Use 1, or Embodiments 1.12, 1.18 or 1.31, wherein R⁴ is independently C₁₋₆-alkyl optionally substituted by one or more halogens.

Embodiment 1.34: Compound For Use 1, or Embodiments 1.12, 1.18 or 1.31, wherein R⁴ is independently C₁₋₄-alkyl optionally substituted by one or more halogens.

Embodiment 1.35: Compound For Use 1, or Embodiments 1.12, 1.18 or 1.31, wherein R⁴ is independently selected from methyl, ethyl, n-propyl, iso-propyl and n-butyl or tert-butyl, preferably methyl, iso-propyl and tert-butyl.

Embodiment 1.36: Compound For Use 1, or any one of Embodiments 1.12, 1.18, 1.25, 1.27, 1.28 and 1.30, wherein R⁴ is independently C₁₋₄-alkyl-C₁₋₄-alkyloxy optionally substituted by one or more halogens.

Embodiment 1.37: Compound For Use 1, or any one of Embodiments 1.13 or 1.19, 1.26, 1.28 to 1.30, wherein both of R⁵ and R⁶ are H.

Embodiment 1.38: Compound For Use 1, or any one of Embodiments 1.13 or 1.19, 1.26, 1.28 to 1.30, wherein at least one of R⁵ and R⁶ is/are independently C₁₋₁₂-alkyl or C₁₋₆-alkyl.C₁₋₆-alkyloxy, optionally substituted by one or more halogens.

Embodiment 1.39: Embodiment 1.38, wherein both R⁵ and R⁶ are independently selected from C₁₋₁₂-alkyl (e.g. C₁₋₃-alkyl, C₁₋₆-alkyl, or C₁₋₄-alkyl), optionally substituted by one or more halogens.

Embodiment 1.40: Embodiments 1.38 or 1.39, wherein R⁵ and/or R⁶ is selected from methyl, ethyl, n-propyl, iso-propyl and n-butyl or tert-butyl, preferably methyl, iso-propyl and tert-butyl, optionally substituted by one or more halogens.

Embodiment 1.41: Embodiments 1.38 to 1.40, wherein R⁵ is methyl and R⁶ is methyl, optionally substituted by one or more halogens.

Embodiment 1.42: Embodiment 1.38, wherein both R⁵ and R⁶ are independently C₁₋₆-alkyl-C₁₋₆-alkyloxy (e.g. C₁₋₄-alkyl.C₁₋₄-alkyloxy, or C₁₋₃-alkyl.C₁₋₃-alkyloxy), optionally substituted by one or more halogens.

Embodiment 1.43: Embodiment 1.38, wherein one of R⁵ and R⁶ is C₁₋₁₂-alkyl and the other one of R⁵ and R⁶ is C₁₋₆-alkyl.C₁₋₆-alkyloxy, optionally substituted by one or more halogens.

Embodiment 1.44: Embodiment 1.43, wherein one of R⁵ and R⁶ is independently C₁₋₃-alkyl and the other one R⁵ and R⁶ is independently selected from C₁₋₄-alkyl.C₁₋₄-alkyloxy, optionally substituted by one or more halogens.

Embodiment 1.45: Embodiment 1.43 or 1.44, wherein one of R⁵ and R⁶ is independently C₁₋₄-alkyl (e.g. methyl, ethyl, n-propyl, iso-propyl and n-butyl or tert-butyl) and the other one of R⁵ and R⁶ is C₁₋₃-alkyl.C₁₋₃-alkyloxy, optionally substituted by one or more halogens.

Embodiment 1.46: Embodiments 1.43 to 1.45, wherein one of R⁵ and R⁶ is independently C₁₋₃-alkyl (e.g. methyl, ethyl, n-propyl, and iso-propyl, preferably methyl) and the other one of R⁵ and R⁶ is independently C₁₋₃-alkyl.C₁₋₃-alkyloxy, optionally substituted by one or more halogens.

Embodiment 1.47: Compound For Use 1, or any one of any one of Embodiments 1.1 to 1.46, wherein Y is a direct bond or —CH═CH—.

Embodiment 1.48: Compound For Use 1, or any one of any one of Embodiments 1.1 to 1.46, wherein Y is —NH— or —O—.

Embodiment 1.49: Compound For Use 1, or any one of any one of Embodiments 1.1 to 1.47, wherein Y is —CH═CH—.

Embodiment 1.50: Compound For Use 1, or any one of any one of Embodiments 1.1 to 1.47, wherein Y is a direct bond.

Embodiment 1.51: Compound For Use 1, or any one of any one of Embodiments 1.1 to 1.46 or 1.48, wherein Y is —NH—.

Embodiment 1.52: Compound For Use 1, or any one of any one of Embodiments 1.1 to 1.46 or 1.48, wherein Y is —O—.

Embodiment 1.53: Compound For Use 1, or any one of any one of Embodiments 1.1 to 1.52, wherein Z is —CH₂—, —C(═CR¹R²)—, —CH(R³)—, —CH(OH)—, or —NR⁴—.

Embodiment 1.54: Compound For Use 1, or any one of any one of Embodiments 1.1 to 1.53, wherein Z is —C(═CR¹R²)— or —CH(R³)—.

Embodiment 1.55: Compound For Use 1, or any one of any one of Embodiments 1.1 to 1.52, wherein Z is —CH₂—.

Embodiment 1.56: Compound For Use 1, or any one of any one of Embodiments 1.1 to 1.52, wherein Z is —C(═CR¹R²)—.

Embodiment 1.57: Compound For Use 1, or any one of any one of Embodiments 1.1 to 1.52, wherein Z is —CR═CR—.

Embodiment 1.58: Compound For Use 1, or any one of any one of Embodiments 1.1 to 1.52, wherein Z is —CH(R³)—.

Embodiment 1.59: Compound For Use 1, or any one of any one of Embodiments 1.1 to 1.52, wherein Z is, —CH(OH)—.

Embodiment 1.60: Compound For Use 1, or any one of any one of Embodiments 1.1 to 1.52, wherein Z is —NR⁴—.

Embodiment 1.61: Compound For Use 1, or any one of any one of Embodiments 1.1 to 1.52, wherein Z is —C(═O)—NR—.

Embodiment 1.62: Compound For Use 1, or any one of any one of Embodiments 1.1 to 1.52, wherein Z is —SO₂—NR—.

Embodiment 1.63: Compound For Use 1, or any one of Embodiments 1.53, 1.54 or 1.56, wherein at least one of R¹ and R² is H.

Embodiment 1.64: Compound For Use 1, or any one of Embodiments 1.53, 1.54, 1.56, or 1.63, wherein R¹ is H and R² is H.

Embodiment 1.65: Compound For Use 1, or any one of Embodiments 1.53, 1.54 or 1.56, wherein at least one of R¹ and R² is C₁-C₆, alkyl, C₁₋₆alkyloxy, or C₁₋₃-alkyl.C₁₋₃-alkyloxy, optionally substituted by one or more halogens.

Embodiment 1.66: Compound For Use 1, or any one of Embodiments 1.53, 1.54, 1.56 or 1.65, wherein at least one of R¹ and R² is C₁-C₄ alkyl, C₁₋₄-alkyloxy, or C₁₋₃-alkyl-C₁₋₃-alkyloxy, optionally substituted by one or more halogens.

Embodiment 1.67: Embodiments 1.65 or 1.66, wherein one of R¹ and R² is H.

Embodiment 1.68: Compound For Use 1, or any one of Embodiments 1.53, 1.54, 1.56, or 1.65 to 1.67, wherein one of R¹ and R² is C₁-C₄ alkyl, optionally substituted by one or more halogens, and the other one of R¹ and R² is H.

Embodiment 1.69: Embodiment 1.68 wherein the C₁-C₄ alkyl selected from methyl, ethyl, n-propyl, iso-propyl and n-butyl or tert-butyl, preferably methyl, ethyl, n-propyl or n-butyl, more preferably methyl or ethyl, optionally substituted by one or more halogens.

Embodiment 1.70: Embodiments 1.67 to 1.69 wherein the one of R¹ and R² which is other than H (i.e. alkyl, alkyloxy or alkyl-alkyloxy) is cis to the X¹—C(═O)—Y—(CH₂)ₙ— moiety of Formula (I), and trans to the —C(═O)—X² moiety of Formula (I).

Embodiment 1.71: Compound For Use 1, or Embodiment 1.57, wherein at least one R of the —CR═CR— group is H.

Embodiment 1.72: Compound For Use 1, or Embodiment 1.57 or 1.71, wherein each R of the —CR═CR— group is H.

Embodiment 1.73: Compound For Use 1, or Embodiment 1.57, wherein each R of the —CR═CR— group is C₁₋₆alkyl, optionally substituted by one or more halogens.

Embodiment 1.74: Compound For Use 1, or Embodiment 1.57 or 1.71, wherein one R of the —CR═CR— group is H and the other R is C₁₋₆alkyl, optionally substituted by one or more halogens.

Embodiment 1.75: Embodiment 1.74, wherein the R which is H is attached to the carbon atom which is also attached to the X¹—C(═O)—Y—(CH₂)ₙ— moiety of Formula (I).

Embodiment 1.76: Embodiment 1.74 or 1.75, wherein each R is in a cis relationship to the other.

Embodiment 1.77: Compound For Use 1, or any one of Embodiments 1.57, 1.73 to 1.76, wherein each R which is alkyl is C₁-C₄ alkyl, optionally substituted by one or more halogens.

Embodiment 1.78: Embodiment 1.61 or 1.62 wherein R is C₁-C₄ alkyl, optionally substituted by one or more halogens.

Embodiment 1.79: Embodiment 1.77 or 1.78, wherein each R which is alkyl is selected from methyl, ethyl, n-propyl, iso-propyl and n-butyl or tert-butyl, preferably methyl, ethyl, n-propyl or n-butyl, optionally substituted by one or more halogens.

Embodiment 1.80: Embodiment 1.53, 1.54 or 1.58, wherein R³ is C₁₋₆alkyl, optionally substituted by one or more halogens.

Embodiment 1.81: Embodiment 1.80, wherein R³ is C₁₋₄-alkyl, optionally substituted by one or more halogens.

Embodiment 1.82: Embodiment 1.81, wherein R³ is selected from methyl, ethyl, n-propyl, iso-propyl and n-butyl or tert-butyl, preferably methyl, ethyl, n-propyl or n-butyl, optionally substituted by one or more halogens.

Embodiment 1.83: Embodiment 1.53, 1.54 or 1.58, wherein R³ is C₁₋₆alkyloxy, optionally substituted by one or more halogens.

Embodiment 1.84: Embodiment 1.83 wherein R³ is C₁₋₄-alkyloxy, optionally substituted by one or more halogens.

Embodiment 1.85: Embodiment 1.53, 1.54 or 1.58, wherein R³ is C₁₋₃-alkyl.C₁₋₃-alkyloxy.

Embodiment 1.86: Embodiment 1.60, wherein R⁴ is C₁₋₂-alkyl, optionally substituted by one or more halogens.

Embodiment 1.87: Embodiment 1.60 or 1.86, wherein R⁴ is independently C₁₋₃-alkyl optionally substituted by one or more halogens.

Embodiment 1.88: Embodiment 1.60, 1.86 or 1.87, wherein R⁴ is independently C₁₋₆-alkyl optionally substituted by one or more halogens.

Embodiment 1.89: Embodiment 1.60, 1.86 to 1.88, wherein R⁴ is independently C₁₋₄-alkyl optionally substituted by one or more halogens.

Embodiment 1.90: Embodiment 1.60, 1.86 to 1.89, wherein R⁴ is independently selected from methyl, ethyl, n-propyl, iso-propyl and n-butyl or tert-butyl, preferably methyl, iso-propyl and tert-butyl.

Embodiment 1.91: Embodiment 1.60, wherein R⁴ is C₁₋₄-alkyl.C₁₋₄-alkyloxy optionally substituted by one or more halogens.

Embodiment 1.92: Compound For Use 1 wherein Y is a direct bond, Z is —CR═CR-n is 0, and wherein at least one R group is H.

Embodiment 1.93: Embodiment 1.92 wherein each R is H,

Embodiment 1.94: Embodiment 1.92 wherein one R is H and the other R is as defined in any one of Embodiments 1.74 or 1.75.

Embodiment 1.95: Embodiment 1.94 wherein the X¹—C(═O)— moiety is trans to the —C(═O)—X² moiety about the Z group.

Embodiment 1.96: Embodiment 1.94 wherein the X¹—C(═O)— moiety is cis to the —C(═O)—X² moiety about the Z group.

Embodiment 1.97: Compound For Use 1, wherein Y is a direct bond, Z is —CH(OH)— and n is 1 or 2.

Embodiment 1.98: Compound For Use 1, wherein Y is a direct bond, Z is —C(═CR¹R²)—, n is 1 or 2, and wherein at least one of R¹ and R² is H, preferably wherein R¹ is H and R² is H.

Embodiment 1.99: Embodiment 1.98 wherein R¹ and R² are as defined in any one of Embodiments 1.64 to 1.70.

Embodiment 1.100: Compound For Use 1, wherein Y is a direct bond or —CH═CH-n is 0, 1 or 2, and is Z —C(═CR¹R²)— or —CH(R³)—.

Embodiment 1.101: Compound For Use 1, or Embodiment 1.100, wherein Y is a direct bond, n is 0 or 1, and Z is —CH(R³)—.

Embodiment 1.102: Embodiment 1.100, wherein R¹ and R² are as defined in any one of Embodiments 1.63 to 1.70.

Embodiment 1.103: Embodiment 1.100 or 1.101, wherein R³ is as defined in any one of Embodiments 1.80 to 1.85.

Embodiment 1.104: any one of Embodiments 1.97 to 1.103, wherein at least one of X¹ and X² is other than —OH.

Embodiment 1.105: any one of Embodiments 1.97 to 1.104, wherein X¹ and X² are as defined in Embodiments 1.22 to 1.30.

Embodiment 1.106: Embodiment 1.105 wherein X¹ and X² are independently selected from —OR⁴ and —NR⁵R⁶, wherein at least one of R⁵ and R⁶, preferably both, are C₁-C₆ alkyl (e.g. methyl, iso-propyl or t-butyl), optionally substituted with one or more halogens; and/or wherein R⁴ is C₁-C₆ alkyl (e.g. methyl, iso-propyl or t-butyl), optionally substituted with one or more halogens.

Embodiment 1.107: Compound For Use 1, or any one of the preceding Embodiments, wherein optional substitution by one or more halogens is by one or more atoms selected from —F, —Cl and —Br, preferably —F.

Embodiment 1.108: Compound For Use 1, wherein the compound of Formula (I) is selected from:

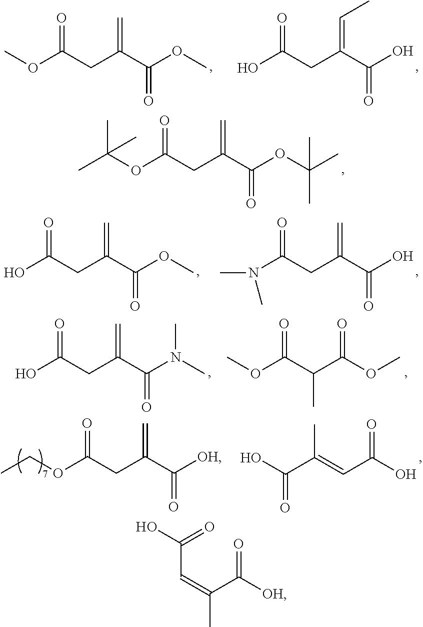

or a pharmaceutically acceptable salt thereof.

Embodiment 1.108: Compound For Use 1, or any one of the preceding Embodiments, wherein the respiratory disease is pulmonary fibrosis.

Embodiment 1.109: Embodiment 1.108, wherein the pulmonary fibrosis is idiopathic pulmonary fibrosis.

Embodiment 1.110: Compound For Use 1, or any one of the preceding Embodiments, wherein the treatment or prevention modifies the metabolic and/or fibrotic phenotype of tissue-resident macrophages, preferably wherein the treatment or prevention increases the metabolic phenotype and/or reduces the fibrotic phenotype of the tissue-resident macrophages.

Embodiment 1.111: Compound For Use 1, or any one of the preceding Embodiments, wherein the treatment or prevention:
a) reduces oxygen consumption rate, maximal respiration and/or spare respiratory capacity of fibroblasts;
b) reduces proliferation of fibroblasts; and/or
c) reduces the wound healing capacity of fibroblasts.

Embodiment 1.112: Compound For Use 1, or any one of the preceding Embodiments, wherein the treatment or prevention results in:
a) an improvement in the fibrosis of the tissue;
b) a decrease in tissue collagen expression, preferably Col3α1, Col1α1 and/or Col4α1;
c) a decrease in tissue fibronectin (Fn1) expression;
d) a decrease in IL-1β expression in fibroblasts obtained from the tissue; and/or
e) a decrease in hydroxyproline levels.

Embodiment 1.113: Compound For Use 1, or any one of the preceding Embodiments, wherein the compound of Formula (I), or pharmaceutically acceptable salt thereof, is used as a part of a combination therapy with another therapeutic agent.

Embodiment 1.114: Compound For Use 1, or any one of the preceding Embodiments, wherein the compound of Formula (I), or pharmaceutically acceptable salt thereof, is delivered in a liposome-based drug delivery system.

Embodiment 1.115: Compound For Use 1, or any one of the preceding Embodiments, wherein the compound of Formula (I), or pharmaceutically acceptable salt thereof, is administered by oropharyngeal inhalation and/or nasal inhalation.

Embodiment 1.116: Compound For Use 1, or any one of the preceding Embodiments, is administered at a dose of about 0.1 mg/kg to about 10 mg/kg.

Embodiment 1.117: Compound For Use 1, wherein the compound is not a compound wherein Y is a direct bond, n is 1, Z —C(═CH₂)—, X¹ is —OR⁴, wherein R⁴ is n-octyl and X² is —OH.

The present invention also provides a pharmaceutical composition comprising a compound of Formula (I), or pharmaceutically acceptable salt thereof, as defined in Compound For Use 1, or any one of Embodiments 1.01 to 1.108 or 1.117, wherein the pharmaceutical composition is adapted for administration by oropharyngeal inhalation and/or nasal inhalation; and/or incorporated into a liposome-based drug delivery system, as for instance described herein.

The present invention also provides methods of treatment or prevention of a respiratory disease characterised by, or involving, lung fibrosis in a subject, said method comprising administering a compound of Formula (I), or a pharmaceutically acceptable salt thereof, as defined in Compound For Use 1, or any one of Embodiments 1.01 to 1.108 or 1.117, to the subject.

### Preparation of Compounds of Formula (I)

As will be appreciated by the skilled person, the compounds of Formula (I) are based on simple dicarbonyl compounds which may be purchased commercially, or readily synthesised by known methods, of which the skilled person is familiar, particularly when taking the benefit of the present disclosure. Reference in that regard is made to: i) ACS Catal. 2016, 6, 2739-2744, which describes synthesis of itaconic acid ester analogues via self-aldol condensation of ethyl pyruvate, catalyzed by hafnium BEA zeolites; ii) J. Org. Chem. 1999, 64, 17, 6411-6417, which describes a general method for the synthesis of enantiomerically pure β-substituted, β-amino acids through α-substituted succinic acid derivatives (scheme 1 thereof, in particular); and iii) Chemistry & Biology 13, 849-856, August 2006, which describes synthesis of 2-oxoglutaric acid and various analogues. Each of these references may be readily adapted for the synthesis of various compounds of Formula (I).

Reference is also made to the below general reaction Schemes 1 to 3, which further illustrate synthetic routes to itaconate analogues and compounds according to Formula (I). As the skilled person will also appreciate, known commercially available organic dicarboxylic acids (including those derived from renewable sources) may be utilised as starting materials in the formation of compounds of Formula (I), for instance, through esterification (Scheme 3) or amination reactions

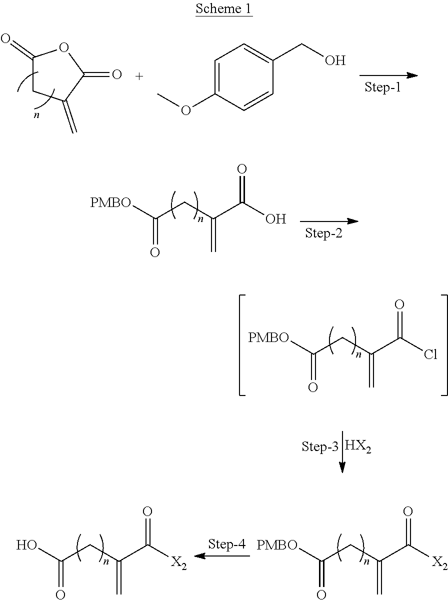

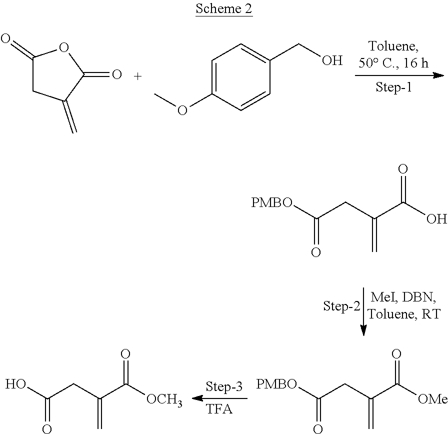

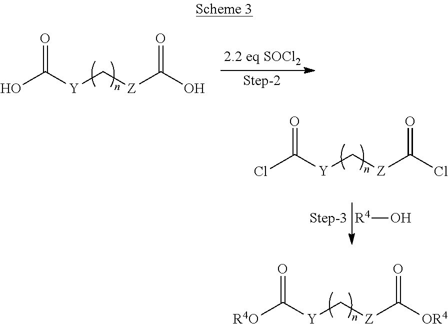

### Inhibition of Succinate Dehydrogenase

Succinate dehydrogenase (SDH) is a heterotetrameric enzyme complex that catalyses the sixth step of the tricarboxylic acid cycle (TCA), i.e. the dehydrogenation of succinate to fumarate. This reaction, through the generation of electrons, couples the TCA with the electron transfer chain. The four subunits are encoded by four genes SDHA, SDHB, SDHC and SDHD. SDHA encodes the main catalytic subunit, a flavoprotein (Fp) containing oxidoreductase.

The terms “inhibit SDH”, “inhibition of SDH” and “SDH inhibitor” as used herein relate to inhibition of the catalytic activity of SDH, and can be used interchangeably with the terms “inhibit SDHA”, “inhibition of SDHA” and “SDHA inhibitor”.

The present invention provides compounds of Formula (I), or pharmaceutically acceptable salts thereof, which may inhibit SDH for use in the treatment or prevention of respiratory diseases characterised by, or involving, lung fibrosis. Such compounds and compositions and drug delivery systems incorporating the same, are described herein.

The present invention relates to direct inhibition of SDH, unless otherwise stated. “Direct inhibition of SDH” as used herein means inhibition of the expression and/or activity of SDH directly, i.e. without any intermediary step. By way of non-limiting example, direct inhibition of SDH may be elicited by competitive or non-competitive inhibitors of the SDH enzyme or by inhibition of a gene or genes encoding the subunits of the SDH enzyme.

“Indirect inhibition of SDH” as used herein means inhibition of the expression and/or activity of SDH indirectly, i.e. through the modulation or delivery of genes/enzymes upstream of SDH and/or through the generation or delivery of intermediaries which directly inhibit SDH. Indirect inhibition may be elicited by upregulating the expression of an enzyme which generates an endogenous direct inhibitor of SDH. By way of non-limiting example, indirect inhibition of SDH may involve increasing the expression and/or activity of aconitate decarboxylase 1 (ACOD1). ACOD1 encodes cis-aconitate decarboxylase (CAD), which catalyses the decarboxylation of cis-aconitate to itaconate. The itaconate produced inhibits SDH. The degree of indirect inhibition may be as defined above.

Expression may be quantified in terms of gene and/or protein expression, and may be compared with the expression of a control (e.g. housekeeping gene or protein). As a non-limiting example, in the context of SDH expression, the actual amount of an SDH gene, mRNA transcript and/or protein, such as the mass, molar amount, concentration or molarity of an SDH gene, mRNA transcript and/or protein, or the number of mRNA molecules per cell in a sample obtained from an individual treated according to the invention and the control may be assessed, and compared with the corresponding value from the control. Alternatively, the expression of an SDH gene and/or protein in a sample obtained from an individual treated according to the invention may be compared with that of the control without quantifying the mass, molar amount, concentration or molarity of the one or more gene and/or protein.

Typically, the control is an equivalent sample in which no inhibition of SDH expression has been effected. As a non-limiting example, in the case where an individual is treated with an agent that inhibits SDH expression, a suitable control would be a different individual to which the compound has not been administered or the same individual prior to administration of the compound. Conventional methods for the assessment of gene and/or protein expression are well known in the art and include RT-qPCR, ELISA, DNA microarray, RNA Seq, serial analysis of gene expression (SAGE) and western blotting.

SDH activity may be quantified in terms of the enzyme's consumption of substrate or production of product, and may be compared with the activity of a control (i.e. recombinant enzyme of known concentration). Conventional methods for the assessment of SDH activity are known in the art and include colorimetric and fluorometric assays.

In the context of the present invention, when referring to (direct or indirect) inhibition of SDH expression and/or activity, the degree of inhibition may be as defined above. Typically, inhibition of SDH resulting in a decrease in SDH activity and/or expression of at least about 5%, at least about 10%, preferably at least about 20%, at least about 25%, at least about 30%, at least about 50%, at least about 75%, up to complete inhibition or abrogation of SDH activity and/or expression.

Compounds of Formula (I), or pharmaceutically acceptable salts thereof, may selectively inhibit SDH, which is typically the case with direct inhibition of SDH. For such compounds which directly inhibit SDH, selectivity may mean that the compounds bind selectively (also referred to interchangeably herein as specifically) with SDH. By “binds selectively”, it will be understood that said compound binds to SDH, with no significant cross-reactivity to any other molecule. Cross-reactivity may be assessed by any suitable method. By way of non-limiting example, cross-reactivity of an compound which inhibits SDH with a molecule other than SDH may be considered significant if the agent binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to SDH. A compound that directly inhibits SDH and that binds selectively to SDH may bind to another molecule at less than 90%, 85%, 80%, 75%, 70%, 65%, 60%, 55%, 50%, 45%, 40%, 35%, 30%, 25% or 20% the strength that it binds to SDH. Preferably, the compound binds to the other molecule at less than 20%, less than 15%, less than 10% or less than 5%, less than 2% or less than 1% the strength that it binds to SDH.

The compounds of Formula (I), or pharmaceutically acceptable salts thereof, may exhibit improved SDH inhibitory activity when compared to itaconate, or may exhibit at least 50% (e.g. at least 60%, 70%, 80% or 90%) of the SDH inhibitory activity of itaconate. The compounds of Formula (I) include modifications to the basic itaconate structure which may, for instance, improve solubility of the compounds in comparison to itaconate, and also disrupt activity of the compounds in the anti-inflammatory pathway that itaconate is known to participate in (e.g. via the KEAP-1-NRF2 axis).

For example, itaconate contains an electrophilic α,β-unsaturated carboxylic acid that is believed to alkylate protein cysteine residues on KEAP-1 by a Michael addition to form a 2,3-dicarboxypropyl adduct, preventing degradation of NRF2 by KEAP-1, and consequently activating a transcriptional anti-oxidant and anti-inflammatory program (Mills et al., 2018, Nature, 556:113-117). The inventors have found that blocking the site of Michael addition in itaconate by introducing substituents at the alkene group of the compound, or by entirely replacing the alkene group with a group inactive to alkylation by KEAP-1 residues, can, surprisingly, still afford compounds which have comparable anti-fibrotic effects. Other modifications, for example, esterification or amination of carboxylic acid groups of itaconate would also be expected to have a negative impact on the anti-inflammatory effects associated with itaconate, yet have been surprisingly found by the inventors to still afford compounds with desirable anti-fibrotic effects and improved solubility profile. This demonstrates further that the anti-fibrotic effects of itaconate derive from a possibly complex anti-fibrotic pathway that appears to be largely divorced from the anti-inflammatory pathway that itaconate is known to interact with. Thus, the design of compounds of Formula (I) would be considered counterintuitive to a person of skill in the art with the knowledge of itaconate's anti-inflammatory activity via the KEAP-1-NRF2 axis, from which its anti-fibrotic effects might also be presumed to at least partly derive.

The compounds of Formula (I), or a pharmaceutically acceptable salt thereof, may be used as direct inhibitors of SDH, in combination with another agent which is an indirect inhibitor of SDH, which may be selected from a nucleic acid (for example, an siRNA, shRNA, or antisense oligonucleotide), antibody or antigen-binding fragment, or an aptamer.

Preferably an agent that indirectly inhibits SDH increases the expression and/or activity of ACOD1. In the context of the present invention, when referring to increasing the expression and/or activity of ACOD1, the degree of increase may be as defined above. Typically, increasing the expression and/or activity of ACOD1 refers to an increase in ACOD1 expression and/or activity of at least about 50%, or at least about 60%, or at least about 70%, or at least about 80%, or at least about 90%, at least about 100% or more.

The disclosure herein regarding determining and/or quantifying the expression and or activity of SDH can also be applied in the context of ACOD1. By way of non-limiting example, ACOD1 activity may be quantified in terms of the enzyme's consumption of substrate or production of product, and may be compared with the activity of a control (i.e. recombinant enzyme of known concentration). Conventional methods for the assessment of ACOD1 activity are known in the art and include colorimetric and fluorometric assays.

An agent acting as an indirect inhibitor of SDH may result in an increase in the level of itaconate within the tissue to be treated. In this context, the level of itaconate encompasses, the actual amount of itaconate, such as the mass, molar amount, concentration or molarity of itaconate (for a set sample size or in individual cells of said sample). Typically, the level of itaconate is determined in a sample obtained from an individual treated according to the invention and the control may be assessed quantitatively, and compared with the corresponding value from the control. Alternatively, the level of itaconate in a sample obtained from an individual treated according to the invention may be compared qualitatively with that of the control i.e. without quantifying the mass, molar amount, concentration or molarity of itaconate.

### Drug Delivery Systems

A compound of Formula (I), or a pharmaceutically acceptable salt thereof, may be delivered by means of a drug delivery system. Drug delivery systems may be used to increase delivery; increase uptake; and/or to increase the efficacy of a compound of Formula (I), or a pharmaceutically acceptable salt thereof, of the invention.

Any appropriate drug delivery system may be used to deliver a compound of Formula (I), or a pharmaceutically acceptable salt thereof, of the invention. Conventional drug delivery systems are known in the art. By way of non-limiting example, appropriate drug delivery systems include liposomes, immunoliposomes, nanoparticles and conjugates. Thus, it would be routine for one of skill in the art to select a suitable drug delivery system. Liposome drug delivery systems are referred to interchangeably herein as liposome-based drug delivery systems.

The skilled person would understand that the choice of drug delivery system may depend on the particular indication to be treated.

As discussed herein, phagocytic cells, such as different populations of macrophages are particularly relevant in the context of fibrosis. Therefore, drug delivery systems (e.g. liposomes or nanoparticles) specifically adapted for phagocytic cells may be used according to the invention. For example, drug delivery systems which specifically or preferentially target phagocytic cells may be used according to the invention. By way of non-limiting example phosphatidyl choline:cholesterol liposomes are a preferred drug delivery system of the invention. Any suitable ratio of phosphatidyl choline:cholesterol may be used in a liposome of the invention, however, liposomes with a 70:30 molar ratio percentage of phosphatidyl choline:cholesterol are particularly preferred. Such liposome drug delivery systems may further be conjugated to antibodies, or antigen binding fragments thereof, which target phagocytic cell-specific cell surface markers. Liposome drug delivery systems may also be glycosyslated, preferably, mannosylated. Drug delivery systems (e.g. liposomes or nanoparticles) may be suited for delivery to phagocytic cells based on their size distribution and/or surface charge, preferably both. Typically, the drug delivery systems will have an average size of between 1 to 5 μm, preferably 1.5 to 2 μm.

### Therapeutic Indications

The compounds of Formula (I), or pharmaceutically acceptable salts thereof, compositions and drug delivery systems as described herein are useful in the treatment of a respiratory disease characterised, or involving, a lung fibrosis.

Fibrosis is a pathological mechanism which occurs in numerous organs and diseases. Fibrosis results from abnormal tissue repair and is associated with persistent and/or severe tissue damage and cellular stress. Failure to adequately contain or eliminate factors triggering fibrosis can exacerbate inflammation and chronic wound-healing responses, resulting in continued tissue damage and inadequate regeneration and, ultimately, fibrosis.

Although fibrosis and inflammation can occur simultaneously, the mechanisms underlying the two processes are distinct. For example, in idiopathic pulmonary fibrosis (IPF) inflammation is often mild and patchy, and clinical trials using anti-inflammatory drugs to treat IPF failed to treat outcomes and instead have been found to increase mortality.

Although differing in aetiology and causative mechanisms, fibrotic diseases all have abnormal and exaggerated accumulation of extracellular matrix (ECM) components, mainly fibrillar collagens. The resulting fibrosis disturbs the normal architecture of affected organs, which ultimately leads to their dysfunction and failure. Due to the common mechanism underlying fibrosis in respiratory diseases, the compounds, compositions and drug delivery systems of the invention are useful in treating lung tissue fibrosis in a range of diseases. The fibrosis itself may be a hallmark of a particular respiratory disease (i.e. the respiratory disease in question is characterised by the presence of fibrosis) or fibrosis may be involved, even where fibrosis itself is not a common characteristic of a particular respiratory disease diagnosis, as, for instance, a result of a chronic respiratory disorder (e.g. certain forms of asthma).

Thus, in some embodiments, the invention relates to the treatment of pulmonary fibrosis. The pulmonary fibrosis may thus be drug induced (e.g. as a result of exposure to amiodarone, nitrofurantoin, chemotherapy, methotrexate etc), radiation induced, environmental induced (hypersensitivity pneumonitis) (e.g. exposure to allergens), autoimmune induced (connective tissue disease-associated interstitial lung disease—CTD-ILD), occupational induced (pneumoconiosis) (e.g. exposure to dust, fibers, fumes, asbestos, coal, silica). In other embodiments, the respiratory disease may also include forms of asthma which have given rise to airway fibrosis (e.g. airway subepithelial fibrosis).

Preferably the invention relates to the treatment of chronic fibrosing interstitial lung disease, even more preferably to the treatment of IPF.

### Treatment Outcomes

“Treatment” according to the present invention may be defined as providing a treatment outcome as defined below. These definitions may apply to therapeutic and prophylactic treatments as described herein.

Treatment may modify the metabolic and/or fibrotic phenotype of Tr-Ms. In particular, “treatment” may be defined as increasing the metabolic phenotype and/or decreasing the fibrotic phenotype of Tr-Ms. An increase in the metabolic phenotype of Tr-Ms may be defined as an increase in the proportion of CD11b⁺/MHCII⁺ Tr-Ms and/or a decrease in the proportion of CD11b⁻/MHCII⁻ Tr-Ms. For example, treatment may: (i) increase the proportion of CD11b⁺/MHCII⁺ Tr-Ms by at least 10%, at least 15%, at least 20% or more; and/or (ii) decrease the proportion of CD11b⁻/MHCII⁻ Tr-Ms by at least 2%, at least 3%, at least 4%, at least 5% or more.

Alternatively or in addition, treatment may modify the metabolic and/or fibrotic phenotype of fibroblasts within the tissue to be treated. In particular, “treatment” may be defined as decreasing the metabolic and/or fibrotic phenotype of fibroblasts within the tissue to be treated. A decrease in the metabolic phenotype of fibroblasts may be defined as a decrease in the OCR, maximal respiration rate and/or spare respirator capacity of the fibroblasts. A decrease in the fibrotic phenotype of fibroblasts may be defined as a decrease in the fibroblast proliferation rate and/or a decrease in the wound healing capacity of the fibroblasts. A decrease in the fibrotic phenotype of fibroblasts may also be defined in terms of the expression of fibrotic markers. By way of non-limiting example, a decrease in the fibrotic phenotype of the fibroblasts may be defined as a decrease in matrix metalloproteinase, TGFβ1 and/or CD71 expression.

Treatment may modify the metabolic and/or fibrotic phenotype of Tr-Ms and fibroblasts. In particular, “treatment” may be defined as: (i) increasing the metabolic phenotype of Tr-Ms; (ii) decreasing the fibrotic phenotype of Tr-Ms; (iii) decreasing the metabolic phenotype of fibroblasts; and or (iv) decreasing the fibrotic phenotype of fibroblasts; within the tissue to be treated. Any combination of (i)-(iv) is encompassed by the present invention. Preferably treatment encompasses all of (i)-(iv).

Treatment according to the invention may result in: (a) an improvement in the fibrosis of the lung tissue; (b) a decrease in tissue collagen expression, preferably Col3α1, Col1α1 and/or Col4α1; (c) a decrease in lung tissue fibronectin (Fn1) expression; (d) a decrease in IL-1β expression in fibroblasts obtained from the lung tissue; and/or (e) a decrease in hydroxyproline levels. Any combination of (a)-(e) is encompassed by the present invention. Preferably treatment encompasses all of (a)-(e).

By way of non-limiting example, treatment may result in: (a) an improvement in lung function; (b) a reduction in the decline of forced vital capacity; (c) preservation or improvement of exercise capacity; (d) a reduction in the progression of fibrosis as quantified by high resolution computed tomography; (e) preservation or improvement of quality of life; and/or (f) an improvement in survival. Any combination of (a)-(f) is encompassed by the present invention. Preferably treatment encompasses all of (a)-(f).

An improvement in lung function may be defined as one or more of (i) an increase in force vital capacity (FVC); (ii) an increase in total lung capacity; and/or (iii) and increase in the transfer capacity of the lung for the uptake of carbon monoxide, as measured by a gas transfer (TLco) test; or (iv) any combination thereof. Any combination of (i)-(iii) is encompassed by the present invention. Preferably treatment encompasses all of (i)-(iii).

Treatment according to the present invention may result in any combination of the treatment outcomes as described herein.

### Therapy

The invention provides a compound of Formula (I), or a pharmaceutically acceptable salt thereof, pharmaceutical compositions and/or drug delivery systems for delivering the same, for use in the treatment or respiratory diseases, characterised by, or involving, lung fibrosis. Said compound of Formula (I) may inhibit SDH, either directly or indirectly.

The term “treat” or “treating” as used herein encompasses prophylactic treatment (e.g. to prevent onset of tissue fibrosis) as well as corrective treatment (treatment of an individual already suffering from tissue fibrosis). Preferably, the term “treat” or “treating” as used herein means corrective treatment. The term “treat” or “treating” encompasses treating both lung tissue fibrosis, symptoms thereof and diseases/disorder associated therewith. In some embodiments the term “treat” or “treating” refers to a symptom of lung tissue fibrosis.

The “treatment” may be defined as providing a treatment outcome as defined herein. For example, the “treatment” may modify: (i) the metabolic and/or fibrotic phenotype of Tr-Ms; and/or (ii) the metabolic and/or fibrotic phenotype of fibroblasts within the lung tissue to be treated; as described herein. In particular, “treatment” may be defined as: (i) increasing the metabolic phenotype and/or decreasing the fibrotic phenotype of Tr-Ms; and/or (ii) decreasing the metabolic and/or fibrotic phenotype of fibroblasts within the tissue to be treated; as described herein.

A compound of Formula (I), or a pharmaceutically acceptable salt thereof, pharmaceutical composition or drug delivery system described herein may be used in the treatment of an individual having Tr-Ms with reduced ACOD1 expression. An individual may be screened for the ACOD1 expression of their Tr-Ms prior to treatment (e.g. using a sample or biopsy of the tissue to be treated), and may be selected for treatment based on the level of expression of the Tr-Ms. In the case of an individual to be treated for pulmonary fibrosis, the tissue sample used to test for Tr-M ACOD1 expression levels may be a bronchoalveolar lavage (BAL) sample. Typically, the level of ACOD1 expression in Tr-Ms comprised in sample (e.g. a BAL sample) obtained from an individual to be treated is reduced compared to the level in a control sample. The control sample may be from an individual that does not have tissue fibrosis (e.g. if BAL samples are used, pulmonary fibrosis).

A compound of Formula (I), or a pharmaceutically acceptable salt thereof, pharmaceutical composition or drug delivery system described herein may be used in the treatment of an individual having reduced levels of itaconate within the lung tissue undergoing fibrosis. An individual may be screened for the level of itaconate in the lung tissue to be treated prior to treatment (e.g. using a sample or biopsy of the tissue to be treated), and may be selected for treatment based on the level of itaconate in the tissue. In the case of an individual to be treated for pulmonary fibrosis, the tissue sample used to test for the level may be a BAL sample. Typically, the level of itaconate in a BAL sample obtained from an individual to be treated is reduced compared to the level in a control BAL sample (e.g. from an individual that does not have pulmonary fibrosis).

A “therapeutically effective amount” is any amount of a compound of Formula (I), or pharmaceutically acceptable salt thereof, or pharmaceutical composition or drug delivery system comprising the same, which, when administered alone or in combination to a patient for treating a respiratory disease characterised by, or involving, lung tissue fibrosis (or preventing further lung tissue fibrosis) or a symptom thereof or a disease associated therewith is sufficient to provide such treatment of the lung tissue fibrosis, or symptom thereof, or associated disease. A “prophylactically effective amount” is any amount of an of a compound of Formula (I), or pharmaceutically acceptable salt thereof, or pharmaceutical composition or drug delivery system comprising the same, which, when administered alone or in combination to an individual inhibits or delays the onset or reoccurrence of lung tissue fibrosis, or a symptom thereof or disease associated therewith). In some embodiments, the prophylactically effective amount prevents the onset or reoccurrence of lung tissue fibrosis entirely. “Inhibiting” the onset means either lessening the likelihood of tissue fibrosis onset (or symptom thereof or disease associated therewith) or preventing the onset entirely.

The terms “subject”, “individual” and “patient” are used interchangeably herein to refer to a mammalian individual. Generally, the individual may be human; in other words, in one embodiment, the “individual” is a human. The individual may not have been previously diagnosed as having lung tissue fibrosis (or symptom thereof or disease associated therewith). Alternatively, the individual may have been previously diagnosed as having tissue fibrosis (or symptom thereof or disease associated therewith). The individual may also be one who exhibits disease risk factors, or one who is asymptomatic for lung tissue fibrosis (or symptom thereof or disease associated therewith). The individual may also be one who is suffering from or is at risk of developing lung tissue fibrosis (or symptom thereof or disease associated therewith).

Administration of a compound of Formula (I), or pharmaceutically acceptable salt thereof, or pharmaceutical composition or drug delivery system comprising the same, of the invention may be by any appropriate route. Non-limiting examples of conventional routes include inhalation; intraperitoneal, intravenous, intra-arterial, subcutaneous, and/or intramuscular injection; infusion; rectal, vaginal, topical and oral administration. In some preferred embodiments, a compound of Formula (I), or pharmaceutically acceptable salt thereof, or pharmaceutical composition or drug delivery system comprising the same, of the invention is administered by inhalation, preferably oropharyngeal inhalation and/or nasal inhalation. In a particularly preferred embodiment, a compound of Formula (I), or pharmaceutically acceptable salt thereof, or pharmaceutical composition or drug delivery system comprising the same, which is administered by oropharyngeal inhalation.

It will be appreciated by one of skill in the art that the appropriate dosage of a compound of Formula (I), or pharmaceutically acceptable salt thereof, or pharmaceutical composition or drug delivery system comprising the same, which, can vary from individual to individual. Determining the optimal dosage will generally involve the balancing of the level of therapeutic benefit against any risk or deleterious side effects. The selected dosage level will depend on a variety of factors including, the route of administration, the severity of the individual's/patient's fibrosis, and the species, sex, age, weight, condition, general health, and prior medical history of the individual/patient. Advantageously, the present inventors have identified suitable dosages of a compound of Formula (I), or pharmaceutically acceptable salt thereof, or pharmaceutical composition or drug delivery system comprising the same, which provide the anti-fibrotic effects claimed. Typically, the compound of Formula (I), or pharmaceutically acceptable salt thereof, is administered at a dose of about 0.1 to 20 mg/kg (i.e. of the active compound). Preferably, a compound of Formula (I), or pharmaceutically acceptable salt thereof, is administered at a dose of about 0.1 to 10 mg/kg, even more preferably at dose of about 5 to 10 mg/kg. In a particularly preferred embodiment, a compound of Formula (I), or pharmaceutically acceptable salt thereof, is administered at a dose of about 5 to 10 mg/kg by oropharyngeal administration.

The frequency of dosing selected may also be dependent on a range of factors. The skilled person will be able to select the most suitable dosing regimen appropriate for the individual. Typically, a compound of Formula (I), or pharmaceutically acceptable salt thereof, or pharmaceutical composition or drug delivery system comprising the same, is administered between about once every three months to about four times per day. For example, a compound of Formula (I), or pharmaceutically acceptable salt thereof, or pharmaceutical composition or drug delivery system comprising the same may be administered once every three months, once per month, twice per month, once per week, twice per week, 3 times per week, 4 times per week, 5 times per week, 6 times per week, once a day, twice a day, 3 times per day, 4 times per day or more. Preferably, a compound of Formula (I), or pharmaceutically acceptable salt thereof, or pharmaceutical composition or drug delivery system comprising the same, is administered about once per day.

A compound of Formula (I), or pharmaceutically acceptable salt thereof, or pharmaceutical composition or drug delivery system comprising the same, of the invention may have a treatment outcome as defined herein within 8-52 weeks (preferably within 36 weeks, more preferably within 24 weeks, even more preferably within 12 weeks) from baseline. Preferably, administration of a compound of Formula (I), or pharmaceutically acceptable salt thereof, or pharmaceutical composition or drug delivery system comprising the same, of the invention may provide a treatment outcome within 36 weeks, more preferably within 24 weeks, even more preferably within 12 weeks.

The treatment outcome may be sustained (e.g. maintained) subsequent to and/or during treatment for several weeks or months or years. A compound of Formula (I), or pharmaceutically acceptable salt thereof, or pharmaceutical composition or drug delivery system comprising the same, of the invention may provide a sustained treatment outcome for at least 5, 10, 12, 16, 18, 20, 22, 24, 38, 32, 36, 40, 52, 78 or 104 weeks. For example, administration of a compound of Formula (I), or pharmaceutically acceptable salt thereof, or pharmaceutical composition or drug delivery system comprising the same, of the invention may provide a sustained treatment outcome for at least 5 weeks, at least 10 weeks, at least 20 weeks, or at least 52 weeks.

A compound of Formula (I), or pharmaceutically acceptable salt thereof, or pharmaceutical composition or drug delivery system comprising the same, of the invention may be used in combination with one or more additional active ingredient or therapeutic, such as another anti-fibrotic agent and/or an anti-inflammatory. By way of non-limiting example, a compound of Formula (I), or pharmaceutically acceptable salt thereof, or pharmaceutical composition or drug delivery system comprising the same, of the invention may be used in combination with pirfenidone and/or nintedanib. Other suitable anti-fibrotic agents which may be used in combination with a compound of Formula (I), or pharmaceutically acceptable salt thereof, or pharmaceutical composition or drug delivery system comprising the same, of the invention include pamrevlumab (an anti-connective tissue growth factor monoclonal antibody), ziritaxestat (also known as GLPG 1690, an autotaxin inhibitor), PRM-151 (a pentraxin-2 recombinant protein and GB0139 (a galectin 3 inhibitor).

The one or more additional active ingredient or therapeutic may be administered sequentially (before or after) the compound of Formula (I), or pharmaceutically acceptable salt thereof, composition or drug delivery system comprising the same, of the invention. The one or more additional active ingredient or therapeutic may be administered simultaneously with a compound of Formula (I), or pharmaceutically acceptable salt thereof, or pharmaceutical composition or drug delivery system comprising the same of the invention.

The invention also provides methods for the treatment or prevention of a respiratory disease characterised by, or involving, lung fibrosis comprising administering a compound of Formula (I), or pharmaceutically acceptable salt thereof, or pharmaceutical composition or drug delivery system comprising the same, of the invention which inhibits SDH.

The invention also provides a compound of Formula (I), or pharmaceutically acceptable salt thereof, or pharmaceutical composition or drug delivery system, comprising the same which inhibits SDH for use in the manufacture of a medicament for the treatment or prevention of a respiratory disease characterised by, or involving, lung fibrosis.

### Pharmaceutical Compositions

Pharmaceutical compositions for use in the invention comprise a compound of Formula (I), or pharmaceutically acceptable salt thereof, together with a pharmaceutically acceptable carrier, such as an excipient, diluent, adjuvant, as well as optionally a immunoregulatory agent and/or antimicrobial compound. Preferably, the composition is adapted for administration by inhalation and particularly oropharyngeal inhalation.

The compound of Formula (I), or pharmaceutically acceptable salt thereof, may be in the form a hydrate or solvate as described herein.

Pharmaceutical compositions comprising a compound of Formula (I), or pharmaceutically acceptable salt thereof, of the invention may further comprise one or more additional active ingredient or therapeutic, such as another anti-fibrotic agent and/or an anti-inflammatory as described herein. The compound of Formula (I), or pharmaceutically acceptable salt thereof, or pharmaceutical composition or drug delivery system comprising the same, of the invention and the one or more additional active ingredient or therapeutic may be provided as a kit of parts.

As described herein, administration of immunogenic compositions, therapeutic formulations, medicaments and prophylactic formulations is generally by conventional routes, with inhalation and particularly oropharyngeal inhalation, being preferred.

Formulation of compound of Formula (I), or pharmaceutically acceptable salt thereof, or pharmaceutical composition or drug delivery system comprising the same, of the invention may therefore be adapted using routine practice to suit the preferred route of administration.

Formulations suitable for distribution as aerosols are preferred, and it would be routine for one of ordinary skill in the art to prepare such formulations.

By way of further non-limiting example, a compound of Formula (I), or pharmaceutically acceptable salt thereof, pharmaceutical compositions or therapeutic/prophylactic formulations and/or medicaments thereof may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules. The compound of Formula (I), or pharmaceutically acceptable salt thereof, or pharmaceutical composition or drug delivery system comprising the same, of the invention may also be formulated as a dry-powder formulation.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(I′-2′-dipalmitoyl-sn-glycero-3-hydroxyphosphoryl oxy)-ethylamine (CGP 19835 A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, ASO1, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

Preferably, the pharmaceutical compositions are adapted for inhalation, particularly oropharyngeal inhalation. The compound of Formula (I), or a pharmaceutically acceptable salt thereof, presently disclosed compounds may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoroinethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of Formula (I), or a pharmaceutically acceptable salt thereof. Capsules and cartridges (made, for example, from gelatin) for use in an inhaler or insufflator may be formulated containing a powder mix of a presently disclosed compound and a suitable powder base such as lactose or starch.

### EXAMPLES

The invention will be further clarified by the following examples, which are intended to be purely exemplary of the invention and are in no way limiting.

### Materials and Methods

### Cell Culture

Primary human lung fibroblasts were isolated from lung resections of patients undergoing lung cancer surgery or lung transplantation performed after informed consent as approved by an external Research Ethics Committee (REC 15/SC0101) according to Royal Brompton Hospital protocol (Royal Brompton & Harefield NHS Foundation Trust, 2016). Fibroblasts from healthy individuals were also purchased from Lonza (Lonza, Basel, Switzerland).

16 Human Bronchial Epithelial cells (16HBE) at passages 10 to 14 (p10-p14) were obtained from ATCC (US). The HBE cells were maintained in minimum essential medium (MEM) media (ThermoFisher, US) that was supplemented with 10% foetal bovine serum (FBS); 1%, 2 mM glutamine and 1% penicillin/streptomycin.

### Mice

All procedures were approved by the United Kingdom Home Office and conducted in strict accordance with the Animals (Scientific Procedures) Act 1986. The Imperial College London Animal Welfare and Ethical Review Body (AWERB) approved this protocol. All surgery was performed under ketamine and sodium pentobarbital anaesthesia and all efforts were made to minimize suffering.

Lung function measurements were performed using the Flexivent system (Scireq, Montreal, Canada). After induction of anaesthesia with an i.p. injection of Pentobarbitone (50 mg/Kg, Sigma, UK) and i.m. injection of Ketamine (100 mg/Kg) (Fortdodge Animal Health Ltd, Southampton, UK), mice were tracheotomised and attached to the Flexivent ventilator via a blunt-ended 19-gauge needle. Mice were ventilated using the following settings; tidal volume of 7 ml/Kg body weight, 150 breaths/minute; positive end-expiratory pressure approximately 2 cm H2O. Standardisation of lung volume history was done by performing two deep inflations. Subsequently, measurements of dynamic resistance, dynamic elastance and dynamic compliance were determined using the snapshot-150 perturbation, a single frequency sinusoidal waveform. Resultant data was fitted using multiple linear regression to the single compartment model to determine the above parameters.

### Flow Cytometry

Cells were stained with near IR fixable live/dead (ThermoFisher) for 10 minutes in PBS prior to staining for extracellular antigens in 1% FBS/2.5% HEPES/0.2% EDTA in PBS for 20 minutes at 4° C. For assessment of mitochondrial superoxide, cells were stained with 5 uM MitoSOX Red (ThermoFisher) in PBS for 10 minutes at 37° C. Cells were then washed and fixed using IC fix kit (eBioscience). All antibodies were purchased from Biolegend. Data was acquired with Fortessa II and cell sorting on Aria III (BD Biosciences) and analysis was performed in Flowjo software, using FMO's for each antibody.

Real Time PCR After treatment, the supernatant liquid was removed from cell cultures and RLT buffer containing 1% beta-mercaptoethanol was added to each well and then was frozen for later analysis. RNA extraction of cells was performed using the RNeasy Plus Mini Kit (QIAGEN), according to the manufacturer's protocol. Samples were defrosted at room temperature and centrifuged. The homogenate was transferred onto RNeasy MinElute spin columns and purified. RNA was reverse-transcribed into cDNA using the High-Capacity cDNA Reverse Transcription Kit (ThermoFisher) according to the manufacturer's instructions. Real-time PCR was performed using fast-qPCR mastermix (QIAGEN) on a Viia-7 instrument (Applied Biosciences) with Taqman primers for human genes, namely acod1, cd163, fn1, IL-1β, mmp1, mmp9 using actb (Life Technologies) as housekeeping gene.

### Seahorse Analysis

Healthy primary human lung fibroblasts (10,000 per well) were seeded in a seahorse plate pre-coated with Cell Tak (BD Biosciences, Oxford, UK). After an overnight incubation, fibroblasts were treated with different itaconate analogues for 1 hour and they were subsequently incubated with or without 2 ng/ml TGF-β1 for 24 hours. XF medium (nonbuffered RPMI containing 2 mM glutamine, 1 mM pyruvate and 10 mM glucose, pH 7.4, Agilent, Santa Clara, USA) was used to measure Oxygen Consumption Rate (OCR) and Extracellular Acidification Rate (ECAR) with the XFp extracellular flux analyser. The Seahorse glycolytic Stress Test (Agilent, Santa Clara, USA) was utilized to measure OCR and ECAR at baseline.

### Synthesis of Itaconate Analogues

Various itaconate analogues were synthesised to demonstrate the effectiveness of the analogues according to the present invention. Itaconate (not of the invention), dimethyl itaconate, 4-octyl itaconate, dimethyl malonate (not of the invention), citraconate, and mesaconate were all purchased and used as received from suppliers without further purification. All commercially available reagents were also used as received. The solvents used were all laboratory grade.

### Example 1: Sodium 3-(dimethylcarbamovl)-3-methvlidenepropanoate—“ICL-2”

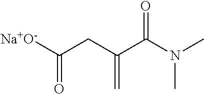

ICL-2 was synthesised according to Scheme 1 hereinabove. Itaconic anhydride and protecting group 4-methoxybenzyl alcohol were added to toluene and heated to 50° C. The solution was heated and stirred for 16 hours. The intermediate was then subjected to SOCl₂ to form an acyl chloride intermediate. 1.2 equivalents of 2 M Me₂NH was cooled in THF before being slowly added to the solution at −78° C. The mixture was then washed with acid at room temperature using 15 equivalents of 4 M HCl in a solvent mixture of dioxane and DCM. The solution was then basified with aqueous NaHCO₃ and separated to remove impurities. The final product was isolated as the sodium salt and verified by ¹H NMR and LCMS.

### Example 2: 3-(dimethylcarbamoyl)-2-methvlidenepropanoic acid—“ICL-PO”

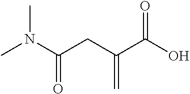

With reference to the reaction scheme below, itaconic anhydride was dissolved in DCM and cooled to −10° C. Cooled dimethylamine was added whilst maintaining a temperature between −10° C. and 0° C. and the reaction vessel was sealed. The mixture was left to react for 1 hour followed by purification to give the final product. The final product was verified by ¹H NMR and LCMS.

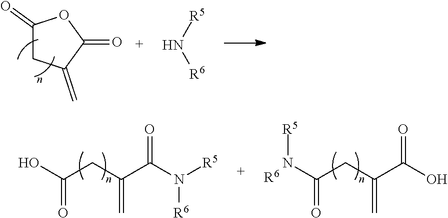

### Example 3: (2E)-2-ethylidenebutanedioic acid (CAS RN 102714-66-9)—“ICL-3”

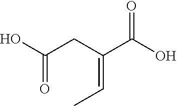

Compound ICL-3 was synthesised in accordance with the below reaction scheme. Ethyl bromoacetate was dissolved in chloroform and (triphenylphosphoranylidene)acetic acid was added. The mixture was refluxed for 24 hours. A Wittig reaction was then performed using acetaldehyde followed by acidification with LiOH to give the final compound. The final product was verified by ¹H NMR and LCMS.

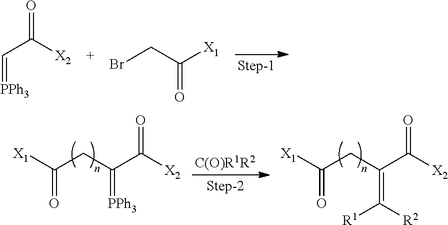

### Example 4a: Bis(2-methyl-2-propanyl)-2-methylenesuccinate (CAS RN 7398-94-9)—“ICL-4”

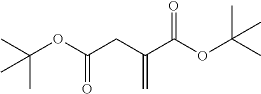 [SMILES: C=C(CC(=O)OC(C)(C)C)C(=O)OC(C)(C)C]

Compound ICL-4 was synthesised directly from itaconate. 3 g of itaconate was dissolved in 15 mL of 1,2-dichloroethane. 6 mL of tert-butanol was added, and the mixture was acidified with 0.5 mL H₂SO₄. The reaction was heated to 70° C. and stirred for 2 hours. The compound was purified using high-performance liquid chromatography (HPLC) and verified via ¹H NMR and LCMS.

### Example 4b: Bis(2-methyl-2-propanyl)-2-methylenesuccinate (CAS RN 7398-94-9)—“ICL-4”

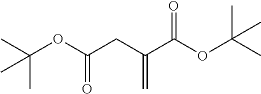 [SMILES: C=C(CC(=O)OC(C)(C)C)C(=O)OC(C)(C)C]

An alternative synthesis method to that of Example 4a reacted 1 g itaconate with 2.2 equivalents of SOCl₂ to form the acyl halide itaconate analogue, as shown in the below reaction scheme. This was followed by quenching with tert-butanol to give compound X. The final product was verified by ¹H NMR and LCMS.

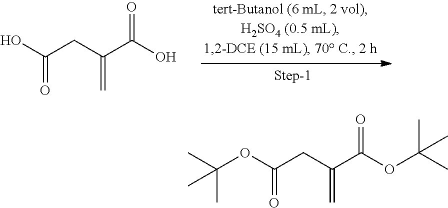

### Example 5: 3-(methoxycarbonyl)but-3-enoic acid, (CAS RN 3377-31-9)—“ICL-6”

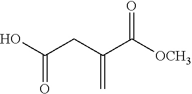

5 g of itaconic anhydride was dissolved in toluene. 6.16 g of protecting group 4-methoxybenzyl alcohol was added and the reaction mixture was heated to 50° C. for 16 hours. The intermediate was then isolated and reacted further with methyl iodide in a solvent solution of DBN and toluene at room temperature to give the methyl ester. The solution was then subjected to trifluoracetic acid to remove the 4-methoxybenzyl alcohol protecting group. The final product was verified by ¹H NMR and LCMS.

### Example 6: The Effect of Itaconate Analogues on Human Bronchial Epithelial Cell Wound Healing

The efficacy of itaconate analogues at limiting the fibrosis related functions of 16 Human Bronchial Epithelial Cells (16HBE) was assessed using standard scratch experiments to assess wound healing and cell proliferation rates.

16HBE cultures were serum-starved, i.e., were subjected to no foetal bovine serum, overnight before being treated with 0.1 mM itaconate (IT), sodium itaconate (NAIT), succinate (SI), dimethyl itaconate (DIMT), and 4-octyl itaconate (4-OI) in MEM media. A DMSO vehicle (V) was used for 4-OI treatment, while all other treatment utilised supplemented MEM.

For the wound healing assays, a scratch was applied along the middle of each well using a standard p10 pipette tip. Top-down images of the wells were taken with a JuLI Stage system (NanoEntek, Korea) at two positions per well every 4 hours for 48 hours (wound healing assay). The images were analysed using ImageJ/Fiji® software and wound healing rates were calculated using an ImageJ/Fiji® plugin by Suarez-Arnedo et al. All wound healing rates were normalised to the starting confluency of each cultured well.

The results of the scratch experiments are shown in FIG. 1. Notably, at both timepoints examined, the treatment of 16HBE with itaconate analogues displayed lower relative wound closure. These findings indicate a reduced healing capacity with the treatments, indicating a potentially protective effect in lung fibrosis.

### Example 7: Itaconate Analogues Control Gene Expression in Human Lung Fibroblast Fibrosis Models

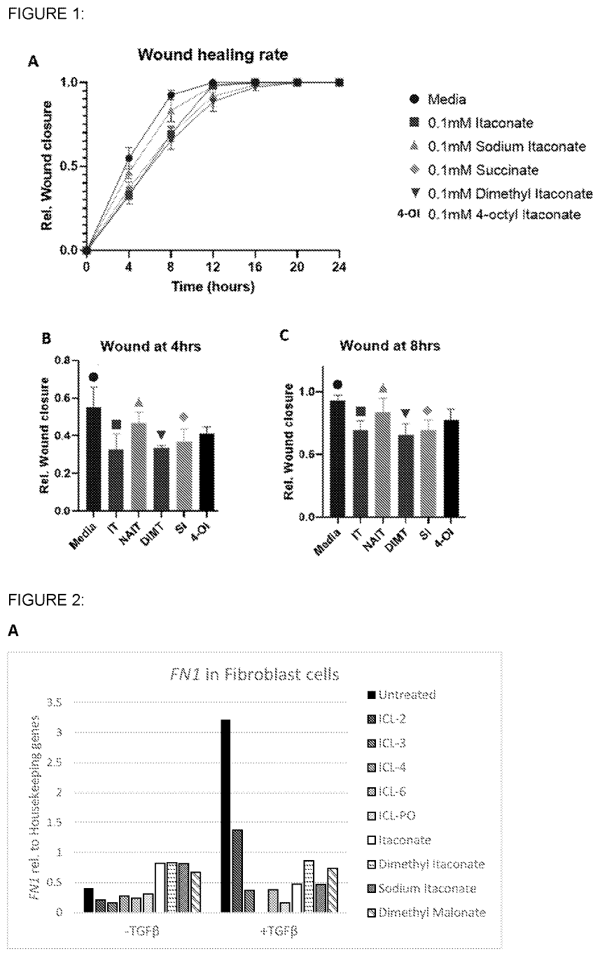

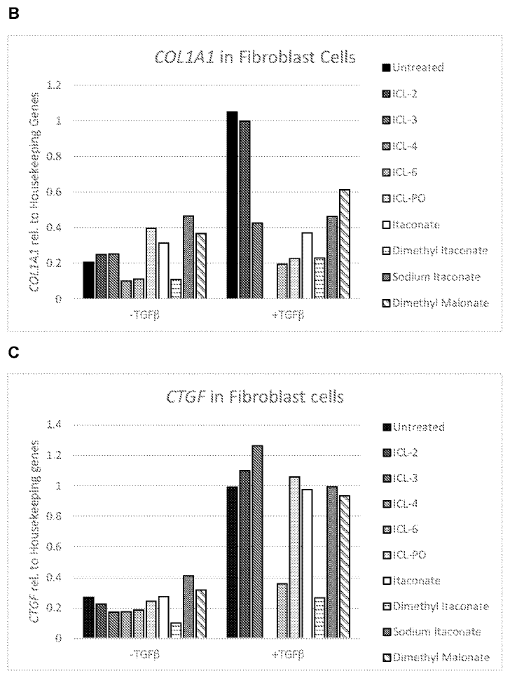

Fibroblasts are the principal effector cell during lung fibrosis and the main source of the excessive extracellular matrix deposition seen during the disease. To explore the anti-fibrotic properties of itaconate analogues in human fibroblast cells, qPCR experiments were undertaken. Human lung fibroblast cells were seeded (10,000 cells/well) in a 48-well plate and were incubated with ICL-PO, ICL-2, ICL-3, ICL-4, ICL-6, itaconate, dimethyl itaconate, sodium itaconate and dimethyl malonate for 1 hour, after which the supernatant liquid was removed. The healthy fibroblasts were then incubated with 2 ng/mL transforming growth factor beta (TGFβ) for 24 hours in order to mimic pulmonary fibrosis. TGFβ is activated in fibrotic diseases and plays a key role in mediating fibrotic tissue. After treatment, RNA extraction of the cells was performed and 10 μg RNA per sample was reverse transcribed into cDNA. RT-PCR was performed with TaqMan primers for Col1α1, connective tissue growth factor (CTGF) and fibronectin (FN1) human genes. The results are shown in FIG. 2—(A) Gene expression of COL1A1 relative to two housekeeping genes (bactin and Beta-2-Microglobulin-averaged to make a ‘pooled’ control); (B) Gene expression of FN1 relative to housekeeping gene; and (C) Gene expression of CTGF relative to housekeeping gene.

The results show that ICL-PO, ICL-2, ICL-3, ICL-4, ICL-6, itaconate, dimethyl itaconate, sodium itaconate and dimethyl malonate all lead to decreased gene expression of Col1α1, CTGF and FN1 in fibrosis models of human lung fibroblast cells relative to untreated cells. These genes are major hallmarks of lung fibrosis and therefore it is shown that these analogues lead to decreased severity of fibrosis.

### Example 8: Effect of Itaconate Analogues on Human Lung Fibroblasts

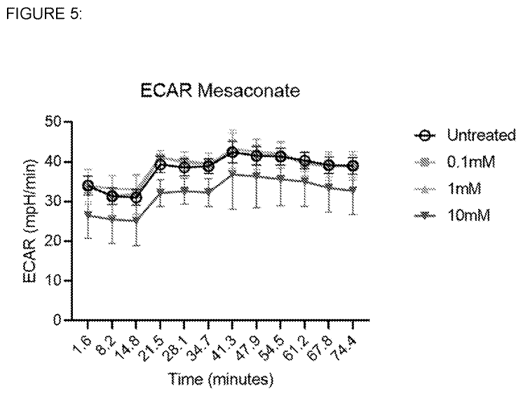

Healthy primary human lung fibroblasts (10,000 per well) were seeded in a seahorse plate pre-coated with Cell Tak (BD Biosciences, Oxford, UK). After an overnight incubation, fibroblasts were treated with different concentrations (0.1 mM, 1 mM and 10 mM) of the itaconate analogues, citraconate or mesaconate, for 1 hour and they were subsequently incubated with or without 2 ng/ml TGF-β1 for 24 hours. XF medium (nonbuffered RPMI containing 2 mM glutamine, 1 mM pyruvate and 10 mM glucose, pH 7.4, Agilent, Santa Clara, USA) was used to measure an Extracellular Acidification Rate (ECAR) with the XFp extracellular flux analyser for the citraconate assay (FIG. 4) or mesaconate assay (FIG. 5). The Seahorse glycolytic Stress Test (Agilent, Santa Clara, USA) was utilized to measure OCR and ECAR at baseline.

ECAR is a measure of lactic acid levels generated by anaerobic glycolysis and is one way in which metabolic activity may be probed. FIGS. 4 and 5 show a concentration dependent reduction in ECAR in response to incubation of human fibroblasts with citraconate and mesaconate respectively. In particular, increasing incubation concentrations of these itaconate analogues lowers ECAR, indicating reduced glycolytic activity in the human lung fibroblasts.

### Example 9: Liposomal Drug Delivery in Bleomycin-Induced Pulmonary Fibrosis in Mice

To assess the efficacy of liposomal drug delivery, the murine bleomycin model of pulmonary fibrosis was utilised for airway macrophages (AM) targeted drug administration. Wild-type (WT) mice were instilled with a single dose of bleomycin via the oropharyngeal route. To assess the lung function, the mice were administered with 50 mg/kg intraperitoneal pentobarbitol and 100 mg/kg intramuscular ketamine to obtain surgical anaesthesia. The tracheas were then cannulated to allow for mechanical ventilation of the mice, using Flexivent™ apparatus. Mice were ventilated with a tidal volume of 7 mL/kg body weight at a ventilation rate of 150 breaths per minute and a positive end-expiratory pressure of approximately 2 cm H₂O, which is similar to normal breathing. Two deep inflations were performed prior to starting measurement to standardise lung volume history.

For drug administration, 10 μL liposomes encapsulating 9.8 mM dimethyl malonate were used. Drug encapsulated or empty control liposomes were diluted 1:4 in PBS and 40 μL was administered via the oropharyngeal route at days: −1, 0, +10, +13, +16, +19 and +21 from the dose of bleomycin.

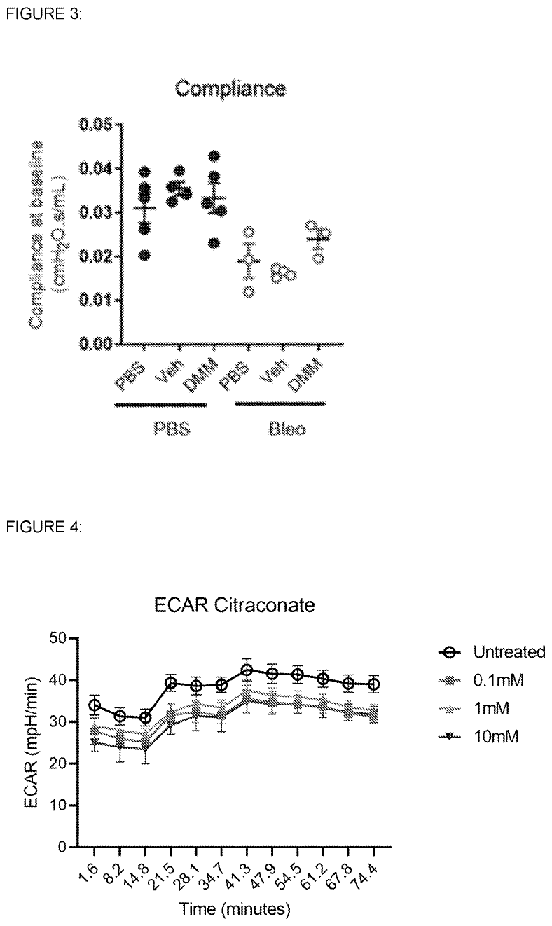

Dynamic resistance, elastance and compliance at baseline was measured using a snapshot-150 perturbation, which is a single frequency sinusoidal waveform. FlexiWare software (Scireq) was used to fit the data to a single compartment model and, using multiple linear regression analysis, airway resistance and pulmonary elastance were calculated to calculate compliance: pressure=(resistance×flow)+(elastance×volume)+fitting constant. The results are shown in FIG. 3.

The results show an overall decrease in lung function in mice subjected to bleomycin, as expected. FIG. 3 also shows an increase in lung function with the therapeutic administration of dimethyl malonate (not of the invention) encapsulated liposomes in the murine bleomycin model of lung fibrosis.

Control and drug loaded liposomes were coupled to a DiD dye to facilitate tracking by flow cytometry. The liposomes were 1.5 to 2 μM in diameter, allowing phagocytosis by AMs. Titration experiments in naïve mice showed that 1:1 and 1:2 dilutions of liposomes with PBS resulted in a high proportion of neutrophils and dendritic cells taking up liposomes, while this markedly decreased at a dilution of 1:4 and still resulted in uptake by over 90% of AMs (data not shown). This showed that AM targeting will greatly improve specific delivery and specificity, allowing reduced amounts of drugs to be utilised and avoiding unwarranted side effect. Targeted delivery of metabolic inhibitor drugs, such as dimethyl malonate, to AMs specifically improves efficacy, sustained drug release and prevents capture by mucus.

### DISCUSSION

The data presented here verify that the anti-fibrotic effects of itaconate can be replicated in structural analogues of that compound, even where, surprisingly, structural modifications are made to the itaconate structure that would be expected to disrupt or preclude anti-inflammatory effects via the KEAP-1-NRF2 axis. Modifications to block the site of Michael addition in itaconate by introducing substituents at the alkene group of the compound (as in the case of compound ICL-3 above), or by entirely replacing the alkene group with a group inactive to alkylation by KEAP-1 residues (as shown for dimethyl malonate and succinate above), can, surprisingly, still afford compounds which have comparable anti-fibrotic effects, as demonstrated herein. Other modifications, for example, esterification or amination of carboxylic acid groups of itaconate (as in the case of compounds ICL-PO, ICL-2, ICL-3, ICL-4 and ICL-6 above), would also be expected to have a negative impact on the anti-inflammatory effects associated with itaconate (compounds ICL-PO and ICL-2, for instance, are also more polar than itaconate, which would reduce cellular uptake compared to itaconate unless there is active transport, which is not known to exist), yet have been surprisingly found by the inventors to still afford compounds with desirable anti-fibrotic effects and improved solubility profile. Structural isomers of itaconate in the form of mesaconate and citraconate (differing only in the location of the internal double bond) have also surprisingly been found by the inventors to exert desirable anti-fibrotic effects, also suggesting that the presence or position of the double bond of itaconate is not essential.

1. A compound of Formula (I), or a pharmaceutically acceptable salt thereof, for use in treating or preventing a respiratory disease characterised by, or involving, lung fibrosis in a subject, 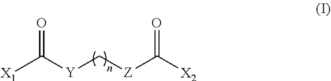
wherein:
Y is a direct bond, —CH═CH—, —NH—, or —O—;
Z is —CH₂—, —C(═CR¹R²)—, —CR═CR—, —CH(R³)—, —CH(OH)—, —NR⁴—, —C(═O)—NR, —SO₂—NR—, —NH— or —O—;
X¹ is —OH, —OR⁴, or —NR⁵R⁶;
X² is —OH, —OR⁴, or —NR⁵R⁶;
R is independently H or a C₁₋₆-alkyl optionally substituted by one or more halogens;
R¹ and R² are independently H, a C₁₋₆-alkyl, C₁₋₆-alkyloxy, or C₁₋₃-alkyl-C₁₋₃-alkyloxy wherein alkyl, alkyloxy and alkyl-alkyloxy groups are optionally substituted by one or more halogens;
R³ is C₁₋₆-alkyl, C₁₋₆-alkyloxy, or C₁₋₃-alkyl-C₁₋₃-alkyloxy optionally substituted by one or more halogens;
R⁴ is independently C₁₋₁₂-alkyl, or C₁₋₆-alkyl-C₁₋₆-alkyloxy optionally substituted by one or more halogens;
R⁵ and R⁶ are independently H, C₁₋₁₂-alkyl, or C₁₋₆-alkyl-C₁₋₆-alkyloxy, wherein alkyl and alkyl-alkyloxy groups are optionally substituted by one or more halogens;
n is 0 to 4
with the following provisos:
1) when Y is —CH═CH—, Z is not —CH₂—;
2) when Y is —CH═CH— and Z is —CR═CR—, n is not 0;
3) when Y is a direct bond, Z is —C(═CH₂)— and n is 1, at least one of X¹ and X² is other than —OH;
4) when Y is a direct bond, Z is —CH₂— and n is 0 or 1, at least one of X¹ and X² is —NR⁵R⁶; and
5) when Y is a direct bond, Z is —CH═CH— and n is 0, at least one of X¹ and X² is —NR⁵R⁶.

2. A compound for use according to claim 1, wherein at least one of X¹ and X² is —OR⁴, or —NR⁵R⁶, preferably wherein at least one of X¹ and X² is —OR⁴.

3. A compound for use according to claim 1, wherein n is 0 to 2, preferably wherein n is 0 or 1.

4. A compound for use according to claim 1, wherein Y is a direct bond or —CH═CH—, preferably wherein Y is a direct bond.

5. A compound for use according to claim 1, wherein Z is —CH₂—, —C(═CR¹R²)—, —CH(R³)—, —CH(OH)—, or —NR⁴—.

6. A compound for use according to claim 1, wherein Z is —C(═CR¹R²)— or —CH(R³)—.

7. A compound for use according to claim 6, wherein Z is —C(═CR¹R²)— and wherein at least one, preferably both, of R¹ and R² is H.

8. A compound for use according to claim 7, wherein one of R¹ and R² is C₁-C₄ alkyl, preferably wherein one of R¹ and R² is methyl, optionally substituted by one or more halogens.

9. A compound for use according to claim 6 wherein Z is —CH(R³)— and wherein R³ is C₁-C₄ alkyl, preferably methyl, optionally substituted by one or more halogens.

10. A compound for use according to claim 1, wherein Z is —CR═CR— and at least one R group, preferably both, is/are H.

11. A compound for use according to claim 10, wherein one R group is C₁-C₄ alkyl, preferably methyl, optionally substituted with one or more halogens.

12. A compound for use according to claim 1, wherein at least one of R⁵ and R⁶, preferably both, are C₁-C₁₂ alkyl, optionally substituted with one or more halogens; and/or wherein R⁴ is C₁-C₁₂ alkyl, optionally substituted with one or more halogens.

13. A compound for use according to claim 12, wherein at least one of R⁵ and R⁶, preferably both, are C₁-C₆ alkyl (e.g. methyl, iso-propyl or t-butyl), optionally substituted with one or more halogens; and/or wherein R⁴ is C₁-C₆ alkyl (e.g. methyl, iso-propyl or t-butyl), optionally substituted with one or more halogens.

14. A compound for use according to claim 1, wherein Y is a direct bond, Z is —CR═CR—, n is 0, and wherein at least one R group, preferably both groups, is/are H, and wherein when one R group is other than H, the R group is C₁-C₆ alkyl (e.g. methyl, iso-propyl or t-butyl), and preferably wherein the X¹—C(═O)— moiety is trans to the —C(═O)—X² moiety.

15. A compound for use according to claim 1, where Y is a direct bond, Z is —CH(OH)—, and n is 1.

16. A compound for use according to claim 1, wherein the compound is selected from: 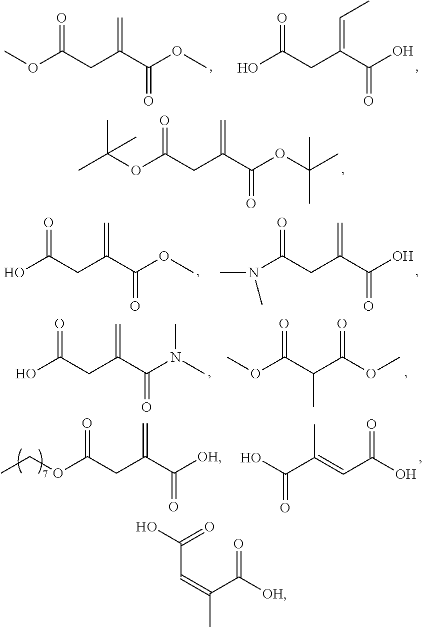
or a pharmaceutically acceptable salt thereof.

17. A compound for use according to claim 1, wherein the respiratory disease is a pulmonary fibrosis, wherein the pulmonary fibrosis is any form of chronic fibrosing interstitial lung disease including idiopathic pulmonary fibrosis.

18. A compound for use according to claim 1, wherein the treatment or prevention modifies the metabolic and/or fibrotic phenotype of tissue-resident macrophages, preferably wherein the treatment or prevention increases the metabolic phenotype and/or reduces the fibrotic phenotype of the tissue-resident macrophages.

19. A compound for use according to claim 1, wherein the treatment or prevention:
a. reduces oxygen consumption rate, maximal respiration and/or spare respiratory capacity of fibroblasts;
b. reduces proliferation of fibroblasts; and/or
c. reduces the wound healing capacity of fibroblasts.

20. A compound for use according to claim 1, wherein the treatment or prevention results in:
a) an improvement in the fibrosis of the tissue;
b) a decrease in tissue collagen expression, preferably Col3α1, Col1α1 and/or Col4α1;
c) a decrease in tissue fibronectin (Fn1) expression;
d) a decrease in IL-1β expression in fibroblasts obtained from the tissue; and/or
e) a decrease in hydroxyproline levels.

21. A compound for use according to claim 1, wherein the compound of Formula (I), or pharmaceutically acceptable salt thereof, is used as a part of a combination therapy with another therapeutic agent.

22. A compound for use according to claim 1, wherein the compound of Formula (I), or pharmaceutically acceptable salt thereof, is delivered in a liposome-based drug delivery system.

23. A compound for use according to claim 1, wherein the compound of Formula (I), or pharmaceutically acceptable salt thereof, is administered by oropharyngeal inhalation and/or nasal inhalation and/or incorporated into a liposome-based drug delivery system.

24. A compound for use according to claim 1, wherein the compound of Formula (I), or pharmaceutically acceptable salt thereof, is administered at a dose of about 0.1 mg/kg to about 10 mg/kg.

25. A pharmaceutical composition comprising a compound of Formula (I), or pharmaceutically acceptable salt thereof, as defined in claim 1, wherein the pharmaceutical composition is adapted for administration by oropharyngeal inhalation and/or nasal inhalation; and/or incorporated into a liposome-based drug delivery system.
